(19) United States
(12) Patent Application Publication
(10) Pub. No.: US 2025/0134422 A1
(43) Pub. Date: May 1, 2025

(54) Systems And Methods for Assessment of Blood Oxygenation

(71) Applicant: Becton, Dickinson and Company, Franklin Lakes, NJ (US)

(72) Inventors: Ilja Guelen, Ravenstein (NL); Jeroen Van Goudoever, Amstelveen (NL)

(21) Appl. No.: 18/934,122

(22) Filed: Oct. 31, 2024

Related U.S. Application Data
(60) Provisional application No. 63/594,899, filed on Oct. 31, 2023.

(51) Int. Cl. A61B 5/1455; A61B 5/00; A61B 5/0205; A61B 5/021; A61B 5/022; A61B 5/1495

(52) U.S. Cl. CPC A61B 5/14552; A61B 5/02116; A61B 5/022; A61B 5/1495; A61B 5/6815; A61B 5/6824; A61B 5/6826; A61B 5/6828; A61B 5/6829; A61B 5/7271; A61B 5/742; A61B 5/0205; A61B 2560/0223; A61B 2560/0462; A61B 2562/0238

(57) ABSTRACT

Systems and methods for assessing arterial oxygen saturation concurrent with performing continuous blood pressure monitoring via the volume clamp method are described. Generally, a cuff system can be configured to fit onto a body appendage. The cuff system can comprise a pressurizable bladder and a photoplethysmograph. Methods can perform arterial oxygen saturation while the artery of the body appendage is in the unloaded state. Minor fluctuations of plethysmogram signal can be utilized along with a computed calibration factor to compute arterial oxygen saturation.

### CROSS REFERENCE TO RELATED APPLICATIONS

The current application claims the benefit of and priority to U.S. Provisional Patent Application No. 63/594,899 entitled “Systems and Methods for Assessment of Blood Oxygenation” filed Oct. 31, 2023, the disclosure of which is hereby incorporated by reference in its entirety for all purposes.

### TECHNOLOGICAL FIELD

The present disclosure is generally related to noninvasive systems and methods for monitoring arterial oxygen saturation.

### BACKGROUND

The proper utilization of many lifesaving medical techniques and treatments depends upon the attending physician obtaining accurate and continually updated information regarding various bodily functions of the patient. Perhaps the most critical information to be obtained by a physician, and that which will often tell the physician a great deal concerning what course of treatment should be immediately instituted, are heart rate, blood pressure, and arterial oxygen saturation.

In settings such as operating rooms and in intensive care units, monitoring and recording these indicators of bodily functions is particularly important. For example, when an anesthetized patient undergoes surgery, it is generally the anesthesiologist's role to monitor the general condition of the patient while the surgeon proceeds with his tasks. If the anesthesiologist has knowledge of the patient's arterial oxygen saturation, heart rate, and blood pressure, the health and condition of the patient's circulatory system can be assessed.

Arterial oxygen saturation is expressed as a percentage of the total hemoglobin in the patient's blood which is bound to oxygen. The hemoglobin which is bound to oxygen is referred to as oxyhemoglobin. In a patient, an arterial oxygen saturation value is above 95% is considered healthy. As blood courses through the capillaries, oxygen is off-loaded into the tissues and carbon dioxide is on-loaded into the hemoglobin. Thus, the oxygen saturation levels in the capillaries is lower than in the arteries. Furthermore, the blood oxygen saturation levels in the veins is even lower, being about 75% in healthy patients. Traditionally, SaO2 is approximated using pulse oximetry (SpO2), due to its non-invasive nature.

### SUMMARY

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In some aspects, the techniques described herein relate to a method for continuous measurement of arterial oxygen saturation.

In some aspects, the techniques described herein relate to a method for continuous measurement of arterial oxygen saturation including: transmitting a first wavelength of light and a second wavelength of light through a body appendage via an emitter within a blood pressure cuff; wherein the first wavelength of light and the second wavelength of light are discrete.

In some aspects, the techniques described herein relate to a method for continuous measurement of arterial oxygen saturation including: sensing light signals of the first wavelength of light and of the second wavelength of light via a light sensor within the blood pressure cuff.

In some aspects, the techniques described herein relate to a method for continuous measurement of arterial oxygen saturation including: receiving, using a health monitoring system, the light signals of the first wavelength of light and of the second wavelength of light.

In some aspects, the techniques described herein relate to a method for continuous measurement of arterial oxygen saturation, wherein the health monitoring system is in connection with the blood pressure cuff.

In some aspects, the techniques described herein relate to a method for continuous measurement of arterial oxygen saturation, wherein the light signals provide a plethysmogram for each of the first wavelength of light and of the second wavelength of light.

In some aspects, the techniques described herein relate to a method for continuous measurement of arterial oxygen saturation including: computing, using the health monitoring system, a ratio between the plethysmogram of the first wavelength of light and the plethysmogram of the second wavelength of light; calibrating, using the health monitoring system, the computed ratio using a calibration factor.

In some aspects, the techniques described herein relate to a method for continuous measurement of arterial oxygen saturation including: computing, using the health monitoring system, arterial oxygen saturation using the calibrated and computed ratio.

In some aspects, the techniques described herein relate to a method, wherein the light signals of the first wavelength are used to monitor blood pressure via a volume clamp method.

In some aspects, the techniques described herein relate to a method, wherein transmitting a first wavelength of light and a second wavelength of light through a body appendage and sensing light signals of the first wavelength of light and of the second wavelength of light via a light sensor are performed while an artery within the body appendage is in an unloaded state.

In some aspects, the techniques described herein relate to a method, wherein the ratio between the plethysmogram of the first wavelength of light and the plethysmogram of the second wavelength of light is computed using: wherein is ratio between the plethysmogram of the first wavelength of light and the plethysmogram of the second wavelength computed in an unloaded state; wherein represents an AC component of the first wavelength when the artery is the unloaded state; represents an AC component of the second wavelength when the artery is the unloaded state; is a DC component of the first wavelength when the artery is the unloaded state; and is a DC component of the second wavelength when the artery is the unloaded state.

In some aspects, the techniques described herein relate to a method, wherein the blood pressure cuff includes an inflatable bladder; wherein the inflatable bladder applies a pressure onto the body appendage such that the artery therein is in the unloaded state.

In some aspects, the techniques described herein relate to a method, wherein the calibration factor is based on a ratio between the plethysmogram of the first wavelength of light and the plethysmogram of the second wavelength of light that is computed while an artery in the body appendage is a loaded state.

In some aspects, the techniques described herein relate to a method, wherein the ratio between the plethysmogram of the first wavelength of light and the plethysmogram of the second wavelength of light is computed using: wherein is the ratio between the plethysmogram of the first wavelength of light and the plethysmogram of the second wavelength; wherein is an AC component of the first wavelength in the loaded state; is an AC component of the second wavelength in the loaded state; is a DC component of the first wavelength in the loaded state; and is a DC component of the second wavelength in the loaded state.

In some aspects, the techniques described herein relate to a method, wherein a calibration is performed for determining a plethysmogram setpoint for blood pressure monitoring while the artery in the body appendage is the loaded state.

In some aspects, the techniques described herein relate to a method, wherein the calibration factor is computed using: wherein is ratio between the plethysmogram of the first wavelength of light and the plethysmogram of the second wavelength computed in a loaded state and wherein is ratio between the plethysmogram of the first wavelength of light and the plethysmogram of the second wavelength computed in an unloaded state.

In some aspects, the techniques described herein relate to a method, wherein is an averaged ratio of two more cardiac cycles.

In some aspects, the techniques described herein relate to a method, wherein is an averaged ratio of two more cardiac cycles.

In some aspects, the techniques described herein relate to a method, wherein the two more cardiac cycles include at least one cycle prior to a loaded cardiac cycle and at least one cycle subsequent to the loaded cardiac cycle; wherein is determined utilizing the loaded cardiac cycle.

In some aspects, the techniques described herein relate to a method, wherein the loaded cardiac cycle is utilized to recalibrate a plethysmogram setpoint for blood pressure monitoring.

In some aspects, the techniques described herein relate to a method further including correcting, using the health monitoring system, the computed ratio for trending plethysmogram signal of the second wavelength of light when the plethysmogram signal of the second wavelength light exhibits trending.

In some aspects, the techniques described herein relate to a method further including correcting, using the health monitoring system, the computed ratio for variations of pressure provided by the blood pressure cuff.

In some aspects, the techniques described herein relate to a method, wherein the first wavelength of light is infrared and the second wavelength of light is red.

In some aspects, the techniques described herein relate to a method, wherein the body appendage is: an arm, a finger, a thumb, a wrist, an ankle, a leg, a toe, an ear, or a temple.

In some aspects, the techniques described herein relate to a method further including displaying the arterial oxygen saturation on a display is connection with the health monitoring system.

In some aspects, the techniques described herein relate to a health monitoring system for continuous arterial oxygen saturation measurement, including: a blood pressure cuff in connection with a computational system.

In some aspects, the techniques described herein relate to a health monitoring system for continuous arterial oxygen saturation measurement, wherein the blood pressure cuff is configured to be fitted onto a body appendage.

In some aspects, the techniques described herein relate to a health monitoring system for continuous arterial oxygen saturation measurement, wherein the blood pressure cuff includes an inflatable bladder, a light emitter, and a light sensor.

In some aspects, the techniques described herein relate to a health monitoring system for continuous arterial oxygen saturation measurement, wherein the light emitter is configured to transmit a first wavelength of light and a second wavelength of light through the body appendage; wherein the light sensor is configured to sense light signals of the first wavelength of light and of the second wavelength of light.

In some aspects, the techniques described herein relate to a health monitoring system for continuous arterial oxygen saturation measurement, wherein the computational system includes a processor and memory.

In some aspects, the techniques described herein relate to a health monitoring system for continuous arterial oxygen saturation measurement, wherein the memory includes one or more applications that include a set of instructions that are configured to direct the processor to: direct the light emitter to transmit the first wavelength of light and the second wavelength of light through a body appendage.

In some aspects, the techniques described herein relate to a health monitoring system for continuous arterial oxygen saturation measurement, wherein the memory includes one or more applications that include a set of instructions that are configured to direct the processor to: direct the light sensor to sense light signals of the first wavelength of light and of the second wavelength of light; receive the light signals of the first wavelength of light and of the second wavelength of light.

In some aspects, the techniques described herein relate to a health monitoring system for continuous arterial oxygen saturation measurement, wherein the light signals provide a plethysmogram for each of the first wavelength of light and of the second wavelength of light.

In some aspects, the techniques described herein relate to a health monitoring system for continuous arterial oxygen saturation measurement, wherein the memory includes one or more applications that include a set of instructions that are configured to direct the processor to: compute a ratio between the plethysmogram of the first wavelength of light and the plethysmogram of the second wavelength of light.

In some aspects, the techniques described herein relate to a health monitoring system for continuous arterial oxygen saturation measurement, wherein the memory includes one or more applications that include a set of instructions that are configured to direct the processor to: calibrate the computed ratio using a calibration factor.

In some aspects, the techniques described herein relate to a health monitoring system for continuous arterial oxygen saturation measurement, wherein the memory includes one or more applications that include a set of instructions that are configured to direct the processor to: compute arterial oxygen saturation using the calibrated and computed ratio.

In some aspects, the techniques described herein relate to a health monitoring system, wherein the light signals of the first wavelength are also used to monitor blood pressure via a volume clamp method.

In some aspects, the techniques described herein relate to a health monitoring system, wherein the instructions that are configured to direct the processor to transmit a first wavelength of light and a second wavelength of light through a body appendage and that sense light signals of the first wavelength of light and of the second wavelength of light via a light sensor are configured to be performed while an artery within the body appendage is in an unloaded state.

In some aspects, the techniques described herein relate to a health monitoring system, wherein the ratio between the plethysmogram of the first wavelength of light and the plethysmogram of the second wavelength of light is computed using: wherein is ratio between the plethysmogram of the first wavelength of light and the plethysmogram of the second wavelength computed in an unloaded state; wherein represents an AC component of the first wavelength when the artery is the unloaded state; represents an AC component of the second wavelength when the artery is the unloaded state; is a DC component of the first wavelength when the artery is the unloaded state; and is a DC component of the second wavelength when the artery is the unloaded state.

In some aspects, the techniques described herein relate to a health monitoring system, wherein the blood pressure cuff includes an inflatable bladder; wherein the inflatable bladder is configured to apply a pressure onto the body appendage such that the artery therein is in the unloaded state.

In some aspects, the techniques described herein relate to a health monitoring system, wherein the calibration factor is based on a ratio between the plethysmogram of the first wavelength of light and the plethysmogram of the second wavelength of light that is computed while an artery in the body appendage is a loaded state.

In some aspects, the techniques described herein relate to a health monitoring system, wherein the ratio between the plethysmogram of the first wavelength of light and the plethysmogram of the second wavelength of light is computed using: wherein is ratio between the plethysmogram of the first wavelength of light and the plethysmogram of the second wavelength; wherein is an AC component of the first wavelength; is an AC component of the second wavelength; is a DC component of the first wavelength; and is a DC component of the second wavelength.

In some aspects, the techniques described herein relate to a health monitoring system, wherein the set of instructions is configured to direct the processor to direct the health monitoring system to perform a calibration for determining a plethysmogram setpoint for blood pressure monitoring while the artery in the body appendage is the loaded state.

In some aspects, the techniques described herein relate to a health monitoring system, wherein the calibration factor is computed using: where wherein is ratio between the plethysmogram of the first wavelength of light and the plethysmogram of the second wavelength computed in a loaded state and wherein is ratio between the plethysmogram of the first wavelength of light and the plethysmogram of the second wavelength computed in an unloaded state.

In some aspects, the techniques described herein relate to a health monitoring system, wherein is an averaged ratio of two more cardiac cycles.

In some aspects, the techniques described herein relate to a health monitoring system, wherein is an averaged ratio of two more cardiac cycles.

In some aspects, the techniques described herein relate to a health monitoring system, wherein the two more cardiac cycles include at least one cycle prior to a loaded cardiac cycle and at least one cycle subsequent to the loaded cardiac cycle; wherein is determined utilizing the loaded cardiac cycle.

In some aspects, the techniques described herein relate to a health monitoring system, wherein the loaded cardiac cycle is utilized to recalibrate a plethysmogram setpoint for blood pressure monitoring.

In some aspects, the techniques described herein relate to a health monitoring system, wherein the set of instructions that are further configured to direct the processor to correct the computed ratio for trending plethysmogram signal of the second wavelength of light when the plethysmogram signal of the second wavelength light exhibits trending.

In some aspects, the techniques described herein relate to a health monitoring system, wherein the set of instructions that are further configured to direct the processor to correct the computed ratio for variations of pressure provided by the blood pressure cuff.

In some aspects, the techniques described herein relate to a health monitoring system, wherein the first wavelength of light is infrared and the second wavelength of light is red.

In some aspects, the techniques described herein relate to a health monitoring system, wherein the body appendage is: an arm, a finger, a thumb, a wrist, an ankle, a leg, a toe, an ear, or a temple.

In some aspects, the techniques described herein relate to a health monitoring system further including a display, wherein the set of instructions that are further configured to direct the processor to display the arterial oxygen saturation.

In some aspects, the techniques described herein relate to a method of operating a noninvasive blood characteristic sensing system including a light emitter, a light sensor, and a inflatable bladder, the method including: (a) encircling a sensing region of a patient appendage with the inflatable bladder; (b) pressurizing the inflatable bladder to a constant pressurization during an open-loop calibration mode, including: (i) emitting light from the light emitter at a first wavelength into the sensing region of the patient appendage; (ii) emitting light from the light emitter at a second wavelength into the sensing region of the patient appendage; (iii) sensing light at the first wavelength from the light emitter at the light sensor, through the patient appendage; (iv) sensing light at the second wavelength from the light emitter at the light sensor, through the patient appendage; (v) generating a first sensed pleth signal based on the sensed light of the first wavelength; (vi) generating a second sensed pleth signal based on the sensed light of the second wavelength; (vii) generating a pleth setpoint based on the first or second sensed pleth signals, wherein the pleth setpoint corresponds to a resting, unstressed arterial volume; (c) generating a closed-loop R for determining arterial oxygen saturation measurement, including: (i) deriving and based on the first sensed pleth signal; (ii) deriving and based on the second sensed pleth signal; (iii) deriving the open-loop R using the formula: open-loop; (d) modulating pressurization of the inflatable bladder in a closed-loop control mode to partially clamp arterial volume within the sensing region via a closed-loop control algorithm, including: (i) emitting light from the light emitter at the first wavelength into the sensing region of the patient appendage; (ii) sensing light at the first wavelength from the light emitter at the light sensor, through the patient appendage; (iii) generating a third sensed pleth signal based on the sensed light of the first wavelength; (iv) emitting light from the light emitter at the second wavelength into the sensing region of the patient appendage; (v) sensing light at the second wavelength from the light emitter at the light sensor, through the patient appendage; (vi) generating a fourth sensed pleth signal based on the sensed light of the second wavelength; (vii) comparing the third or fourth sensed pleth signals with the pleth setpoint to generate a closed-loop error signal; (viii) modulating pressurization of the inflatable bladder responsive to the closed-loop error signal; (e) generating a closed-loop R for determining arterial oxygen saturation measurement, including: (i) deriving and based on the third sensed pleth signal; (ii) deriving and based on the fourth sensed pleth signal; (iii) deriving the closed-loop R using: closed-loop; (f) generating a calibrated R based on closed-loop R and a calibration factor, including: (i) calculating the calibration factor using: wherein is a ratio between a plethysmogram of the first wavelength of light and the plethysmogram of the second wavelength computed during the open-loop calibration mode and wherein is ratio between the plethysmogram of the first wavelength of light and the plethysmogram of the second wavelength computed in the closed-loop control algorithm; and (g) computing arterial oxygen saturation using the calibrated R and a pulse oximetry calibration curve.

In some aspects, the techniques described herein relate to a method, wherein and/or are averaged ratios of two more cardiac cycles.

In some aspects, the techniques described herein relate to a method, wherein the two more cardiac cycles of include at least one cycle prior to a loaded cardiac cycle and at least one cycle subsequent to the loaded cardiac cycle.

In some aspects, the techniques described herein relate to a method, wherein is determined utilizing the loaded cardiac cycle.

In some aspects, the techniques described herein relate to a health monitoring system for continuous arterial oxygen saturation measurement, including: a blood pressure cuff in connection with a computational system, wherein the blood pressure cuff is configured to be fitted onto a body appendage; wherein the blood pressure cuff includes an inflatable bladder, a light emitter, and a light sensor; wherein the light emitter is configured to transmit a first wavelength of light and a second wavelength of light through the body appendage; wherein the light sensor is configured to sense light signals of the first wavelength of light and of the second wavelength of light; wherein the computational system includes a processor and memory; wherein the memory includes one or more applications that include a set of instructions that are configured to direct the processor to: (a) encircle a sensing region of a patient appendage with the inflatable bladder; (b) pressurize the inflatable bladder to a constant pressurization during an open-loop calibration mode, including: (i) emitting light from the light emitter at a first wavelength into the sensing region of the patient appendage; (ii) emitting light from the light emitter at a second wavelength into the sensing region of the patient appendage; (iii) sensing light at the first wavelength from the light emitter at the light sensor, through the patient appendage; (iv) sensing light at the second wavelength from the light emitter at the light sensor, through the patient appendage; (v) generating a first sensed pleth signal based on the sensed light of the first wavelength; (vi) generating a second sensed pleth signal based on the sensed light of the second wavelength; (vii) generating a pleth setpoint based on the first or second sensed pleth signals, wherein the pleth setpoint corresponds to a resting, unstressed arterial volume; (c) generate a closed-loop R for determining arterial oxygen saturation measurement, including: (i) deriving and based on the first sensed pleth signal; (ii) deriving and based on the second sensed pleth signal; (iii) deriving the open-loop R using the formula: open-loop; (d) modulate pressurization of the inflatable bladder in a closed-loop control mode to partially clamp arterial volume within the sensing region via a closed-loop control algorithm, including: (i) emitting light from the light emitter at the first wavelength into the sensing region of the patient appendage; (ii) sensing light at the first wavelength from the light emitter at the light sensor, through the patient appendage; (iii) generating a third sensed pleth signal based on the sensed light of the first wavelength; (iv) emitting light from the light emitter at the second wavelength into the sensing region of the patient appendage; (v) sensing light at the second wavelength from the light emitter at the light sensor, through the patient appendage; (vi) generating a fourth sensed pleth signal based on the sensed light of the second wavelength; (vii) comparing the third or fourth sensed pleth signals with the pleth setpoint to generate a closed-loop error signal; (viii) modulating pressurization of the inflatable bladder responsive to the closed-loop error signal; (e) generate a closed-loop R for determining arterial oxygen saturation measurement, including: (i) deriving and based on the third sensed pleth signal; (ii) deriving and based on the fourth sensed pleth signal; (iii) deriving the closed-loop R using: closed-loop; (f) generate a calibrated R based on closed-loop R and a calibration factor, including: (i) calculating the calibration factor using: wherein is a ratio between a plethysmogram of the first wavelength of light and the plethysmogram of the second wavelength computed during the open-loop calibration mode and wherein is ratio between the plethysmogram of the first wavelength of light and the plethysmogram of the second wavelength computed in the closed-loop control algorithm; and g) compute arterial oxygen saturation using the calibrated R and a pulse oximetry calibration curve.

In some aspects, the techniques described herein relate to a health monitoring system, wherein and/or are averaged ratios of two more cardiac cycles.

In some aspects, the techniques described herein relate to a health monitoring system, wherein the two more cardiac cycles of include at least one cycle prior to a loaded cardiac cycle and at least one cycle subsequent to the loaded cardiac cycle

In some aspects, the techniques described herein relate to a health monitoring system, wherein is determined utilizing the loaded cardiac cycle.

Other features and advantages of the present disclosure will become apparent from the following detailed description, taken in conjunction with the accompanying drawings which illustrate, by way of example, the principles of the disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the disclosure.

FIGS. 1A and 1B provide views of an example of a blood pressure cuff system on a finger of patient. FIG. 1A provides a perspective view. FIG. 1B provides a cross-sectional view.

FIG. 2A provides a schematic of a tissue system and light signal traversing therethrough.

FIG. 2B provides an example of a plethysmogram depicting DC and AC components of the light signal.

FIG. 3 provides an example of a blood pressure chart and corresponding plethysmogram.

FIG. 4 provides an example of a method for performing continual arterial oxygen saturation measurements.

FIG. 5 provides an example of a method for performing continuous arterial oxygen saturation measurements.

FIG. 6A provides an example of a trending plethysmogram.

FIG. 6B provides an example of a chart of arterial oxygen saturation measurements comparing detrended measurements with original measurements.

FIG. 7 provides an example of a health monitoring system for performing arterial oxygen saturation measurement concurrently with volume-clamp blood pressure monitoring.

### DETAILED DESCRIPTION

Continuous transportation of oxygen to the cells of the body is essential to understanding the well-being of the patient. Nearly all of the oxygen transported from the lungs to the rest of the body is carried by hemoglobin stored in the erythrocytes or red blood cells. As hemoglobin releases carbon dioxide and combines with oxygen its color changes from cyan to a bright red. Arterial oxygen saturation is expressed as a percentage of the maximum oxygen which the arterial blood can carry. An oxygen saturation level of about 95%-98% is considered normal in most patients.

Pulse oximetry is a standard test to measure oxygen saturation of arterial blood. A classical pulse oximeter utilizes a clip-on device that clips onto a body appendage (e.g., finger). A photoplethysmograph (PPG) within the devices generally emits and captures two wavelengths of light (one infrared, one red) to measure the absorbance of the signals of the two wavelengths of light during arterial pulsation to compute an oxygen saturation.

Another important hemodynamic measurement to understanding the well-being of the patient is blood pressure. One method to compute continuous blood pressure is the volume clamp method. The volume clamp method generally measures arterial blood pressure on a body appendage (e.g., finger) utilizing an inflatable cuff and PPG. The pressure in the cuff is adjusted to keep the diameter of the artery constant (the unloaded state), in which the diameter is determined via a light source and light sensor of the PPG (closed-loop). The pressure within the inflatable cuff represents the arterial pressure of the finger artery. In some embodiments, the pressure applied to the body appendage can be at a plurality of pressures at a plurality of times. For example, a first pressurization can be based on a blood pressure of the body appendage. In some embodiments, the blood pressure is configured to be equal to the blood pressure of the body appendage. The system can apply a second pressurization to the body appendage. The second pressurization lower than the first pressurization. For example, in some embodiments, the second pressurization is zero or some nominal amount of pressure.

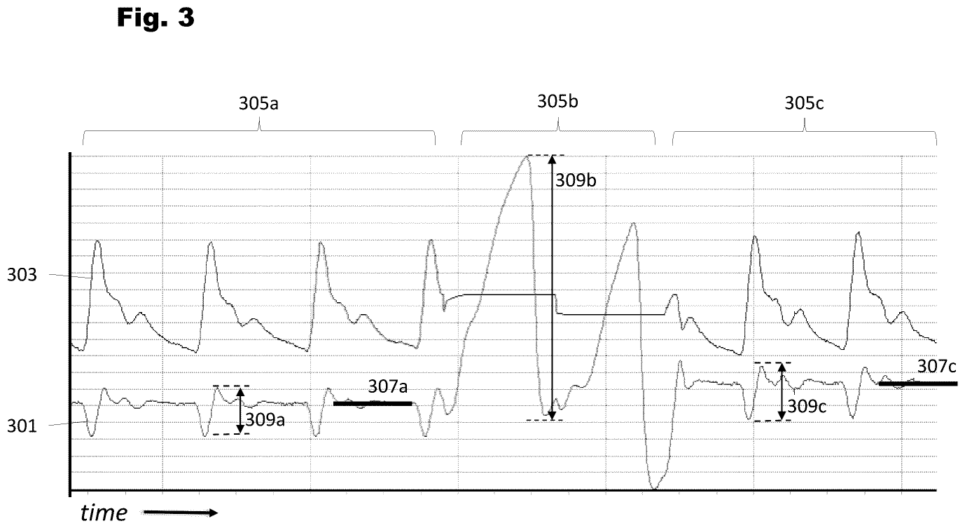

In some embodiments, the system can apply a constant pressurization to the body appendage. This may be useful, for example, to calibrate the system. Calibration can help improve accuracy and/or consistency of blood pressure and/or pulse wave measurements. Using calibration, the system can to periodically adjust its baseline, accounting for changes in the appendage's vascular conditions, such as blood volume shifts, temperature changes, and/or finger movement. During such calibration, the system can monitor changes in blood pressure within the appendage, which may result in significant changes in measurements (see, e.g., section 305b of FIG. 3). During calibration, even minor sensor or measurement drifts over time can be corrected by resetting the system's measurement baseline. Thus, after calibration, a cuff (e.g., cuff 20) may be better able to apply just enough pressure to match an arterial blood pressure without causing discomfort or excessive occlusion. Additionally or alternatively, the calibration can help the system distinguish between physiological signal and noise. As shown in FIG. 3, the system can transmit light at a plurality of wavelengths through the body appendage while the cuff applies a constant pressurization to the appendage. The constant pressurization applied to the body appendage may be advantageously applied for at least a full cardiac cycle. A full cardiac cycle can include at least a systolic and diastolic pressure. In some embodiments, the constant pressurization may be no greater than an atmospheric or ambient pressure. For example, it may be valuable to measure pulse oximetry when no pressure is applied to the cuff from a pump.

It can be valuable to determine a pulse oximetry during a calibration step. For example, a patient's pulse oximetry may be vital during an emergency situation, and during the calibration step, it may be dangerous to the patient to fail to obtain pulse oximetry data, which can be a serious flaw in modern systems and methods.

Classical systems and methods for concurrently performing pulse oximetry and volume-clamp blood pressure measurement typically utilized two different PPG systems, each PPG system on a unique body appendage. This is because the blood pressure cuff of the volume clamp system is configured to keep the diameter of the artery constant and pulse oximetry systems rely on changes of arterial diameter as measured by the PPG to compute oxygen saturation. In other words, classical methods of pulse oximetry cannot be reliably performed when the artery is maintained in the unloaded state.

Here, systems and methods are described for performing both pulse oximetry and volume-clamp blood pressure measurement utilizing a single PPG system on one body appendage. A PPG system can comprise components for performing pulse oximetry (e.g., emitter of two light wavelengths) and can work in conjunction with components for performing volume clamp blood pressure measurements (e.g., emitter of at least one light wavelength and blood pressure cuff that continuously adjusts to keep the arterial diameter in the unloaded state). It is now understood that minor fluctuations in plethysmogram signal in the artery in the unloaded state can be utilized to compute oxygen saturation using a compensation factor that can be determined and continually updated when the artery is a loaded state (e.g., when determining a plethysmogram setpoint during blood pressure measurement calibration). Some implementations of the systems and methods can include other corrective factors that may improve the oxygen saturation measurement.

As utilized throughout the disclosure, an artery is in an unloaded state when the artery is kept at a constant diameter and in a loaded state when the artery is not kept at a constant diameter. In some situations, a blood pressure cuff may apply some pressure on the artery when in a loaded state, but does not prohibit volume changes of the artery.

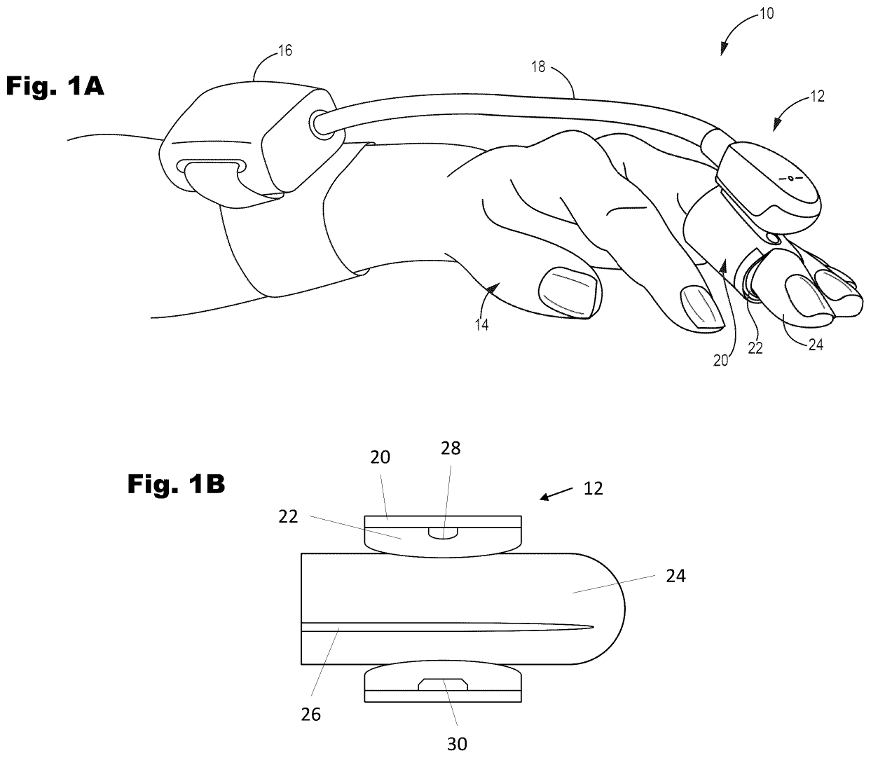

FIG. 1A provides a simplified perspective view sensor system 12 attached to hand 14. FIG. 1B is a schematic view of sensor system 12 on a finger. Although the sensor is depicted as finger, it should be understood that volume clamp blood pressure measure and arterial pulsation can be performed on any body appendage capable of being receiving a blood pressure cuff. Examples of body appendages that can be utilized include (but are not limited to) an arm, a finger, a thumb, a wrist, an ankle, a leg, a toe, an ear, a temple, etc.

As shown in FIG. 1A, sensor system 12 is a non-invasive hemodynamic sensor capable of generating arterial blood pressure measurements through volume clamping and arterial blood oxygen saturation. Sensor system 12 can include housing 16, connector 18, cuff 20, and pressurizable bladder 22. In the illustrated embodiment, cuff 20 is a ring or similar structure surrounding or bracketing finger 24 of hand 14, while housing 16 is a wrist-mounted device coupled to cuff 20 via connector 18. In the most general case, however, sensor system 12 can differ substantially from the layout illustrated in FIG. 1A. Sensor system 12 can, for example, include multiple separate connectors 18 between elements attached to finger 24 (e.g. cuff 20), and/or can relocate housing 16 to other locations (e.g. integrated with cuff 20, or separately disposed at a peripheral location). Further, cuff 20 can be configured to fit on a variety of body appendages, including an arm, a finger, a thumb, a wrist, an ankle, a leg, a toe, an ear, a temple, etc. In the illustrated example, cuff 20 surrounds a sensing region of a finger 24 of hand 14. At least one artery 26 passes through the sensing region, and generally other tissue may be present (e.g., muscle, skin, connective tissue, vein, capillaries). Cuff 20 also anchors pressurizable bladder 22, which can for example be an expandable annular fluid bladder fed by a fluid line included within connector 18, or from another source. Generally, fluid utilized to inflate a bladder can be air. In the most general case, however, pressurizable bladder 22 can be any sort of mechanism suited to apply pressure to finger 24 based on control as described below. Sensor system 12 and hand 14 together make up combined physical system 10 (sometimes referred to as a plant or plant system) responsive both to changes in the patient and change in control of sensor system 12.

As shown in FIG. 1B, cuff 20 includes light emitter 28 and light sensor 30. Light emitter 28 is configured to emit light through the sensing region, which is configured to be sensed by light sensor 30. In some examples, one or more of the wavelengths of light emitted by light emitter 28 can fall within the visible-to-infrared spectrum. Light sensor 30 is configured to detect both overall received light amplitude and specific received light amplitudes of the discrete wavelengths (or discrete bands of wavelengths) emitted by light emitter 28. In some implementations, light emitter 28 comprises a single emission source (e.g., light emitting diode (LED)) that can provide two or more discrete wavelengths of light (or bands of wavelength of light). The term “discrete” means that the wavelengths have a clearly identifiable peak wavelength. In some embodiments, the discrete wavelengths may have a full width at half maximum (FWHM) of less than about 200 nm, less than about 100 nm, less than about 80 nm, less than about 50 nm, less than about 30 nm, less than about 20 nm, less than about 10 nm, less than any value therein, or fall within a range having any value therein as endpoints. In some implementations, light emitter 28 comprises at least two emission sources (e.g., at least two diodes) such that two or more discrete wavelengths of light (or bands of wavelength of light) can be emitted simultaneously. Generally, when at least two emission sources are utilized, the light emission sources can be provided near one another (e.g., within 0.5 mm) to yield similar light pathways, but any configuration can be utilized. In some implementations, light emitter 28 and light sensor 30 can be situated on opposite sides of cuff 20, such that light travels through the sensing region of finger 24 from light emitter 28 to light sensor 30. More generally, however, scattering of light from light emitter 28 inside tissue of finger 24 allows light emitter 28 and light sensor 30 to be effective even when not disposed on opposite sides of cuff 20, e.g. when located proximate one another.

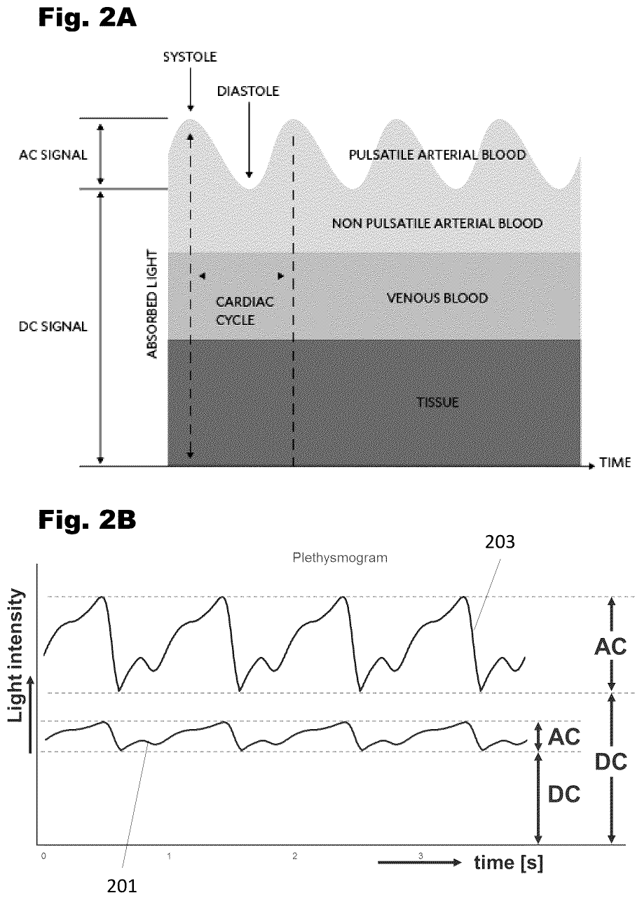

Overall light received at light sensor 30 from transmission by emitter 28 can be used to generate and/or may be referred to hereinafter as a plethysmogram signal. The plethysmogram signal can be used as a proxy for inverse arterial volume within the sensing region, with a reduction in received light corresponding to an increase in arterial volume (see FIG. 2A and accompanying description). During normal blood flow, two arteries of the finger and connected capillaries pulsate, expanding (with systolic pressure) and relaxing (with diastolic pressure) in volume over the course of each heartbeat cycle. Greater arterial volume increases absorption of emitted light, reducing the fraction of emitted light received at light sensor 30.

Light emitter 28 can emit a discrete or fixed known spectrum of light across a range of wavelengths (e.g. a range of or including primarily visible-to-infrared wavelengths) within which absorption differs detectably across material compositions of interest. In some implementations, light emitter 28 can emit a range of wavelengths exclusively within the red and infrared band (600 nm to 1000 nm). In some implementations, a broader range of light including higher energy visible light and/or microwave band light can be used (e.g. 400 nm to 1100 nm). In the most general case, light emitter 28 produces known amplitudes of light at a range of wavelengths broad enough to distinguish absorption spectra associated with at least two parameters including but not limited to blood oxygen saturation, total hemoglobin, percentage methemoglobin, or percentage carboxyhemoglobin.

Light emitter 28 can include a single source in some embodiments. For example, in some embodiments, the light emitter 28 includes a light emitting diode (LED) that can generate one or more wavelengths of light (e.g., discrete wavelengths of light). Additionally or alternatively, the light emitter 28 can transmit a first wavelength of light at the first time and a second wavelength of light at a second time. Use of a single light emitter (e.g., single LED) may be particularly helpful in promoting the portability of the device and/or reducing an energy demand applied to a power source (e.g., battery) of the system. It may be advantageous to track sequential (e.g., consecutive) cardiac cycles. Accordingly, the system may cause the light emitter 28 to emit the first and second wavelengths within a threshold time of each other. For example, a difference between the second time and the first time may be less than a full cardiac cycle, less than half a cardiac cycle, and/or less than a quarter of a cardiac cycle.

Sensor system 12 clamps arterial volume within the sensing region by a pneumatically pressurizable bladder 22 through actuation of a valve (or any fluid flow metering element) and thereby modulating air pressure provided to bladder 22 via connector 18. Examples of valves that can be utilized include (but are not limited to) a servo valve or a piezo pump. Sensor system 12 can clamp arterial volume by any approach that applies a known pressure to the sensing region of finger 24. When performing volume-clamp blood pressure monitoring, the air pressure provided to bladder 22 modulates to counteract arterial pulsations such that the arterial volume within sensing region remains relatively constant. The light emitter 28 can emit light through a portion of the cuff 20 (e.g., bladder 22). The portion can include a material that is translucent. The material may additionally or alternatively be flexible, durable, and/or biocompatible. The material can include a thermoplastic polyurethane (TPU), a silicone, latex, nitrile rubber, polyvinyl chloride (PVC), and/or some other flexible materials. By using a translucent material, the system may be able to create a more compact sensor system 12, thus increasing portability and/or reducing an energy demand on the power source. For more on types of potential light emitters, 28, bladders 22, and cuffs 20, see International Patent Application No. PCT/US2024/019599, the disclosure of which is incorporated herein by reference.

Provided in FIG. 2A is a schematic of tissue of a body appendage and how light from an emitter traverses therethrough. The body appendage comprises tissue, venous blood, and arterial blood. When in an unclamped state (as shown in FIG. 2A), arterial blood comprises nonpulsatile blood and pulsatile blood. For each cardiac cycle, the pulsatile blood volume increases to a maximum at systole and decreases to a minimum during diastole. A light signal can traverse through the body appendage and is absorbed by the various components of the body appendage as it passes therethrough. More light is absorbed during systole due to the increase of arterial blood volume.

As can be seen in FIG. 2A, a portion of the light signal that is absorbed is constant regardless of the moment in the cardiac cycle. Principally, the light that is absorbed by the tissue, the venous blood, and the nonpulsatile arterial remains constant over time. This constant light signal is referred to as the DC signal. A portion of light signal will vary as dependent on arterial blood pulsation as related to cardiac cycles. Light signal absorption is greatest at systole and least at diastole. The variable light signal is referred to as the AC signal.

The DC signal and the AC signal can be utilized to compute blood oxygen saturation. Provided in FIG. 2B is plethysmogram of two wavelengths of light. A first wavelength of light 201 is absorbed greater and provides less light signal than a second wavelength of light 203. Generally, longer wavelengths of light (e.g., IR light) display better transmission through tissue than shorter wavelengths of light (e.g., red light), but the amount of signal received will vary based on light absorption of particular wavelengths and the intensity of light provided by the emitter. Using the DC and AC signals of two wavelengths of light (λ1 and λ2), a ratio (R) can be computed as follows:

$$R = \frac{( \frac{{AC}_{λ1}}{{DC}_{λ1}} )}{( \frac{{AC}_{λ2}}{{DC}_{λ2}} )} \qquad ( Eq . No . 1 )$$

Ratio R can be plotted against oxygen saturation values (e.g., SpO2) as determined experimentally to yield a calibration curve. The calibration curve can be used in analysis of arterial oxygen saturation as determined utilizing a PPG fitted upon a body appendage. It may be valuable to ensure that the ratio R can be accurately determined. It may be helpful to ensure that the AC value (e.g., for the first and/or second wavelengths) is greater than some minimum threshold value. Accordingly, in some embodiments, the system can ensure that the AC signal is greater than a threshold minimum value. Additional information can be found, for example, in International Patent Application No. PCT/US2024/011383, the disclosure of which is incorporated herein by reference.

As noted previously, arterial oxygen saturation is not reliably determined when the artery is in the unloaded state, such as when the body appendage is clamped to continuously measure blood pressure via the volume clamp method. An example of a hemodynamic chart depicting plethysmogram signals 301 and cuff pressure 303 is provided in FIG. 3. The hemodynamic chart is a typical chart for continuous measurement of blood pressure via the volume clamp method, which utilizes a light signal of a single wavelength (or a single band of wavelengths). The chart can be divided into three sections: a first section 305a that depicts blood pressure measured at first plethysmogram setpoint, a second section 305b that depicts a calibration phase for calibrating the plethysmogram setpoint, and a third section that depicts blood pressure measured at a second plethysmogram setpoint.

As can be seen in section 305b, the calibration phase (open-loop) for calibrating the plethysmogram setpoint holds the cuff pressure 303 constant in a step wise fashion in order to determine the appropriate plethysmogram setpoint. Holding the cuff pressure 303 constant can be advantageously applied for at least a full cardiac cycle. This may help ensure that a full and/or accurate calibration can be achieved. Additionally or alternatively, in some embodiments the constant cuff pressure 303 may be no greater than an atmospheric or ambient pressure. For example, it may be valuable to measure pulse oximetry when no pressure is applied to the cuff from a pump (e.g., when an associated pump is off or disconnected). Recalibration of plethysmogram setpoint can be performed repeatedly to ensure blood pressure readings are accurate over time. During continuous blood pressure monitoring, plethysmogram setpoint recalibration can be repeated based on a temporal window (e.g., every 5 minutes), a number of cardiac cycles, (e.g., 300 cardiac cycles), a signal suggesting the plethysmogram setpoint may need recalibration, or any other indicator. Notably, during the calibration phase, the artery is the loaded state, meaning that the cuff pressure does not keep the arterial blood volume constant. Thus, the plethysmogram signal 301 has high amplitude during the calibration phase, reflecting pulsation of arterial blood through the sensing region. The repeated calibration phase can be utilized to for determining arterial oxygen saturation in a continual or a continuous fashion. As indicated elsewhere herein, it may be valuable to determine a pulse oximetry during a calibration step. Accordingly, in some embodiments, the system can transmit a plurality of wavelengths of light through the appendage (e.g., through the cuff) to generate a corresponding set of plethysmograms. For more on for calibrating the plethysmogram setpoint, see U.S. Pat. No. 4,510,940, the disclosure of which is incorporated herein by reference.

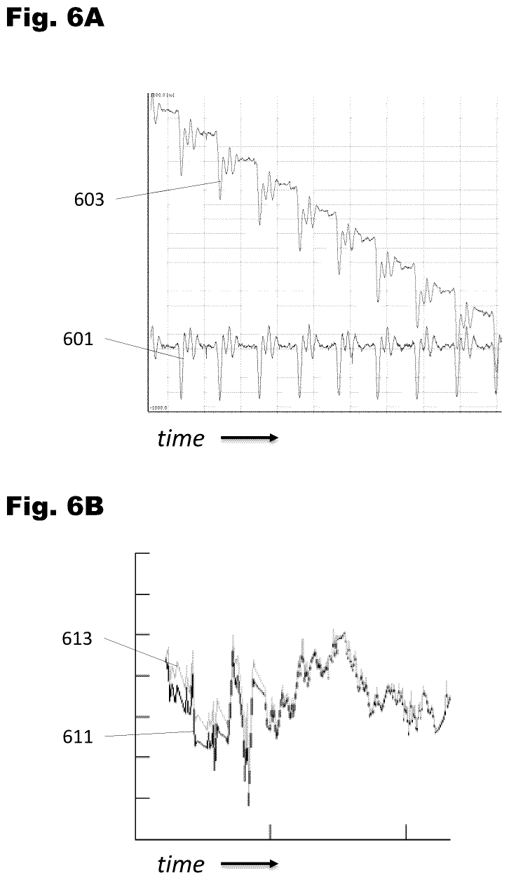

In some contexts, it may be valuable to calibrate a plurality of setpoints. Accordingly, in some embodiments, the cuff can transmit light during a plurality of time windows. For example, the cuff can transmit light at a plurality of wavelengths during a first time window. This light can be sensed and converted to one or more plethysmogram signals. The plethysmogram signals can be used to generate corresponding plethysmograms. Based on the plethysmogram(s), the system can generate a first plethysmogram setpoint. During a different time window, the cuff can transmit light (e.g., at a plurality of wavelengths) during a second time window to generate additional plethysmograms and a corresponding second plethysmogram setpoint. FIG. 6A shows an example of a plurality of plethysmograms. One or more of the setpoints can be used to calculate one or more ratios between plethysmograms. By identifying a difference between plethysmograms over time (e.g., between the first and second time windows), the system can calculate or otherwise determine a degree of drift (e.g., trend) between the plethysmogram setpoints. In some embodiments, this drift can be used to update a determination of arterial oxygen saturation. For example, a drift that is lower over time may cause the system to compensate by increasing a calculated arterial oxygen saturation (e.g., linearly compensate, geometrically compensate, etc.).

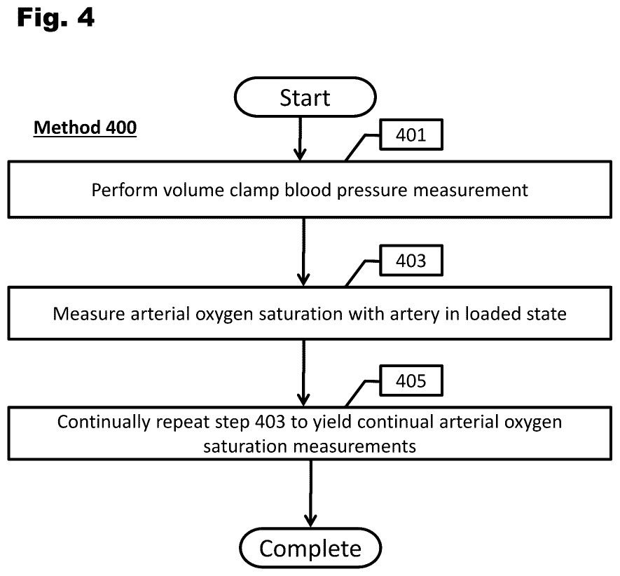

Provided in FIG. 4 is an example of a method for continual measurement of arterial oxygen saturation. Method 400 can be performed using a sensor system configured to perform volume clamp blood pressure measurement and further configured with a PPG to emit and sense two discrete wavelengths of light (or two discrete wavelength bands of light), such as the example sensor system described in references to FIGS. 1A and 1B.

Method 400 can perform (401) volume clamp blood pressure measurement on a body appendage of a patient. Accordingly, a sensor system comprising a cuff with a pressurizable bladder and a PPG can be fit onto a body appendage of a patient. Upon fitting the sensor system, an initial calibration can be performed to determine the plethysmogram setpoint. Using the pressurizable bladder, an artery of the body appendage can be fixed to an unloaded state such that its volume is constant. The variation in the pressure of the pressurizable bladder represents the variation of the blood pressure of the artery, which can be continuously monitored. Recalibration of the plethysmogram setpoint can be iteratively performed.

The method 400 can additionally or alternatively include applying (e.g., using an inflatable bladder of the cuff) a first pressurization to a body appendage. The first pressurization can be based on a blood pressure of the body appendage. For example, the blood pressure may be configured to be equal to the blood pressure of the body appendage. The system can apply (e.g., using the inflatable bladder) a second pressurization to the body appendage. The second pressurization may be lower than the first pressurization. For example, in some embodiments, the second pressurization is zero or some nominal amount of pressure. Applying different pressurizations can allow a practioner, for example, to determine a blood pressure and/or a saturation of arterial oxygen during an interim off-state of the cuff (e.g., between readings, after a first reading, etc.). A first pressurization may be applied for at least a first amount of time (e.g., greater than 5 seconds, greater than 30 seconds, greater than 1 minute, etc.), and/or a second pressurization may be applied for at least a second amount of time.

Method 400 can further measure (403) arterial oxygen saturation with artery in a loaded state. Any instance of when the artery is in a loaded state can be utilized. In some instances, arterial oxygen saturation is measured concurrently with recalibration of the plethysmogram setpoint. When the artery is in a loaded state, two discrete wavelengths of light (or two discrete wavelength bands of light) can be transmitted through the sensing region of the body appendage to measure DC and AC for each wavelength (or each wavelength band). The ratio of DC and AC for each wavelength (or each wavelength band) can be computed. In some implementations, the ratio of DC and AC for each wavelength (or each wavelength band) is computed using Eq. No. 1. The ratio of DC and AC for each wavelength (or each wavelength band) and a calibration curve can be utilized to determine arterial oxygen saturation.

Method 400 can continually repeat (405) step 403 to yield continual arterial oxygen saturation measurements. In some implementations, arterial oxygen saturation is determined each time the plethysmogram setpoint is recalibrated. In some implementations, arterial oxygen saturation is iteratively determined. In some implementations, arterial oxygen saturation is determined when the artery is in a loaded state but not concurrently with recalibration of plethysmogram setpoint for blood pressure monitoring.

Returning back to FIG. 3, section 305a and section 305c show that the plethysmogram signal is constrained to a setpoint (see plethysmogram setpoint 307a and plethysmogram setpoint 307c). Notably, even though the plethysmogram signal is set to be constant, there is minor variation in plethysmogram signal, which may arise due to arterial wall movement during systole. This minor variation in plethysmogram signal can be utilized to compute arterial oxygen saturation but is not very reliable as it is more prone to noise due to the smaller AC component and the difference in light paths between the wavelengths (or two wavelength bands) used to compute the AC component, resulting in greater error between readings and among patients. It has been found, however, the minor variation in plethysmogram signal can be made reliable by computing the AC component when the artery is the loaded state (e.g., during calibration of the plethysmogram setpoint) and computing a calibration factor to calibrate the AC component when the artery is in the unloaded state. The AC component is also referred to as the Δd for change of in a difference of AC component signals during a cardiac cycle.

FIG. 3 depicts examples of a Δd for a few cardiac samples, which can be computed in the artery in the unloaded state (Δd_{Unl}) and in the loaded state (Δd_{Loa}). In particular, Δd_{Unl} 309a is an example of a difference of AC component signals during a cardiac cycle within section 305a and Δd_{Unl} 309c is an example of a difference of AC component signals during a cardiac cycle within section 305c. Δd_{Loa} 309b is an example of a difference of AC component signals during a cardiac cycle within section 305b, which is equivalent to the AC component computed in Eq. No. 1. In these examples, Δd_{Unl} and Δd_{Loa} are each defined as the difference of an AC component signal maximum and an AC component signal minimum, but any change of AC component features can be utilized. Examples of Δd_{Unl} definitions include (but are not limited to) the difference of an AC component signal maximum and an AC component signal minimum, the difference of the plethysmogram setpoint and an AC component signal minimum, the difference of an AC component signal maximum and the plethysmogram setpoint, the difference of an AC component average and an AC component signal minimum, the difference of an AC component signal maximum and an AC component average, the difference of a value of an AC component signal prior to a minimum and an AC component signal minimum, the difference of a value of an AC component signal prior to a maximum and an AC component signal maximum, etc.

The AC signal with the artery in the unloaded state relies on minor signal fluctuation. A servo gain control can be utilized to gain the AC signal, however the gain of the AC signal must not be gained too much or the blood pressure measurements will have error. Accordingly, in many implementations, a servo gain control is utilized to ensure that the AC signal with the artery in the unloaded state has enough fluctuation to compute arterial oxygen saturation but does not provide error to the continuous blood pressure monitoring. For more on servo gain control, see U.S. Provisional Appl. No. 63/479,721, the disclosure of which is incorporated herein by reference.

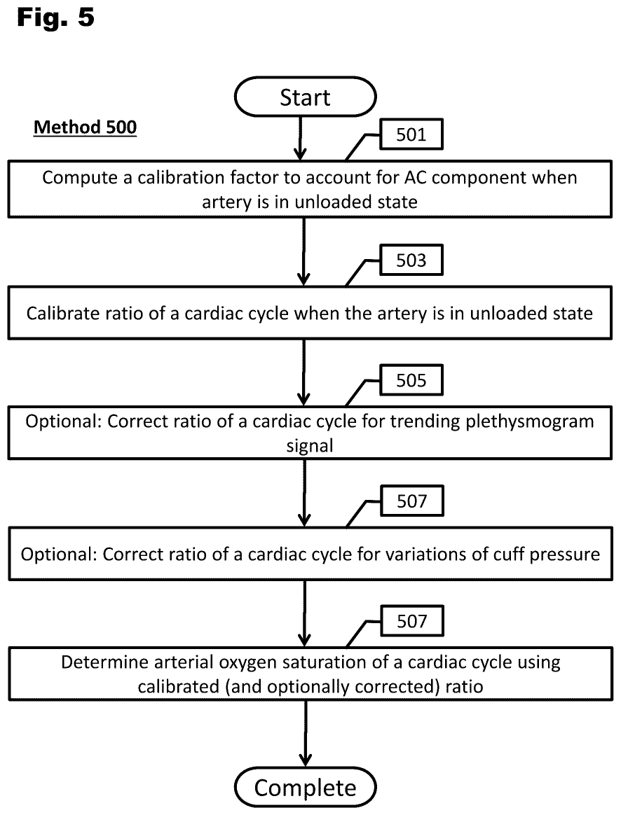

Provided in FIG. 5 is an example of a method for continuous measurement of arterial oxygen saturation. Method 500 can be performed using a sensor system configured to perform volume clamp blood pressure measurement and further configured with a PPG to emit and sense two discrete wavelengths of light (or two discrete wavelength bands of light), such as the example sensor system described in references to FIGS. 1A and 1B. In some implementations, the continuous measurement of arterial oxygen saturation occurs concurrently with the continuous measurement of blood pressure via the volume clamp method.

Method 500 can compute (501) a calibration factor to account for AC component when artery is in an unloaded state, such as when blood pressure is measured via the volume clamp method. Because an AC component is less reliable when the artery is in an unloaded state, determination of arterial oxygen saturation can benefit from using a calibration factor that is determined based on an AC component when the artery is a loaded state. Accordingly, Method 500 takes advantage of ratio R computed when the artery is in the loaded state during plethysmogram setpoint calibration, which can be computed as provide Eq. No. 1 and can be referred to as R_{Loa}. Additionally, ratio R can be computed when the artery is the unloaded state as follows:

$${R}_{Unl} = \frac{( \frac{Δ {d}_{Unl \_λ1}}{{DC}_{Unl \_λ1}} )}{( \frac{Δ {d}_{Unl \_λ2}}{{DC}_{Unl \_λ2}} )} \qquad ( Eq . No . 2 )$$

where Δd_{Unl_λ1} represents the AC component of a first wavelength when the artery is the unloaded state and Δd_{Unl_λ2} represents the AC component of a second wavelength when the artery is the unloaded state.

To compute a calibration factor, the ratio R when the artery is in the loaded state (R_{Loa}) is compared with the ratio R when the artery is in the loaded state (R_{Unl}). Any means for calibrating the ratio R when the artery is in the loaded state using the ratio R when the artery is in the loaded state can be implemented. In some implementations, a calibration factor is computed as follows:

$$Cal\_factor = \frac{{R}_{Loa}}{{R}_{Unl}} \qquad ( Eq . No . 3 )$$

In some implementations, the ratio R when the artery is in the loaded state (R_{Loa}) is an averaged ratio (or otherwise combined ratio) of two or more cardiac cycles. For example, section 305b of FIG. 3 consists of two cardiac cycles and thus these two ratios can be combined in any statistical means for combining values.

In some implementations, the ratio R when the artery is in the unloaded state (R_{Unl}) is an averaged ratio (or otherwise combined ratio) of two or more cardiac cycles. Any two or more cardiac cycles when the artery is in the unloaded state can be combined. In some implementations, the two or more unloaded cardiac cycles to be combined are proximate (or otherwise nearby) loaded cardiac cycles utilized to calculate. For example, the ratio R of one or more cardiac cycles within section 305a and/or section 305c of FIG. 3 can be utilized with ratio R of one or more cardiac cycles within section 305b to compute a calibration factor.

In some implementations, to compute a calibration factor, the ratio R when the artery is in an unloaded state (R_{Unl}) is a combined ratio comprising one or more unloaded cardiac cycles prior to a loaded cardiac cycle. In some implementations, the ratio R when the artery is in an unloaded state (R_{Unl}) is a combined ratio comprising one or more unloaded cardiac cycles subsequent to a loaded cardiac cycle. In some implementations, the ratio R when the artery is in an unloaded state (R_{Unl}) is a combined ratio comprising one or more unloaded cardiac cycles prior to a loaded cardiac cycle and one or more unloaded cardiac cycles subsequent to the loaded cardiac cycle are combined.

In some implementations, a system for continuous measurement of arterial oxygen saturation repeatedly terminates maintaining the artery in an unloaded state in order to recalculate a calibration factor. A calibration factor can be recalculated based on a temporal window (e.g., every 5 minutes), a number of cardiac cycles, (e.g., 300 cardiac cycles), a signal suggesting the calibration factor may need to be recomputed, or any other indicator. Generally, an artery can be transitioned from an unloaded state to a loaded state and a calibration factor is computed. In some implementations, a calibration factor is recalculated when a plethysmogram setpoint is recalibrated for continuous blood pressure monitoring. In some implementations, a calibration factor is continually recalculated. In some implementations, a calibration factor is continually recalculated each time a plethysmogram setpoint is recalibrated for continuous blood pressure monitoring. In some implementations, a calibration factor is continually recalculated every other time a plethysmogram setpoint is recalibrated (or any other periodicity associated with plethysmogram setpoint recalibration). In some instances, a calibration factor is computed independent of recalibration of a plethysmogram setpoint for continuous blood pressure monitoring. In some instances, an artery is transitioned from an unloaded state to a loaded state and a calibration factor is computed but recalibration of a plethysmogram setpoint for continuous blood pressure monitoring is not performed. In some instances, an artery is transitioned from an unloaded state to a loaded state to recalibrate a plethysmogram setpoint for continuous blood pressure monitoring and but a calibration factor is not computed and instead a previously computed calibration factor is utilized.

Method 500 further calibrates (503) the ratio R of a cardiac cycle when the artery is in the unloaded state (R_{Unl}). The means for calibrating the ratio R of a cardiac cycle may depend on how the calibration factor is computed. In some implementations, when the calibration factor is computed using Eq. No. 3, the ratio of an unloaded cardiac cycle is calibrated by multiplying the ratio with the calibration factor.

Method 500 optionally corrects (505) the ratio of an unloaded cardiac cycle for trending plethysmogram signal. As stated previously, only one wavelength of light signal is needed to perform continuous blood pressure monitoring whereas measurement of arterial oxygen saturation utilizes two wavelengths of light signal. By combining continuous blood pressure monitoring with measurement of arterial oxygen saturation, one the wavelengths of light used for with measurement of arterial oxygen saturation can be the wavelength of light used for continuous blood pressure monitoring. Based on the principles of continuous blood pressure monitoring via the volume clamp method, the amplitude of the wavelength of light utilized for blood pressure monitoring maintains a fairly stable amplitude of light signal. The amplitude of the other wavelength of light that is not utilized in continuous blood pressure monitoring may trend, especially at the outset of signal acquisition.

Provided in FIG. 6A is an example of plethysmogram signals of two wavelengths of light utilized in a dual system for performing continuous blood pressure monitoring and measurement of arterial oxygen saturation. A first plethysmogram signal 601 of a wavelength of light maintains a steady amplitude, as this is the wavelength of light that is utilized to establish a plethysmogram setpoint for continuous blood pressure monitoring. A second plethysmogram signal 603 of a wavelength of light has a trending amplitude, which is common for when the wavelength of light is not utilized to establish the plethysmogram setpoint for continuous blood pressure monitoring. The trending of the plethysmogram signal can impair the accuracy of arterial oxygen saturation measurements because it results in changes of the AC component.

Method 500 optionally corrects the computed ratio by detrending the trending plethysmogram signal. Any method for detrending can be utilized. Examples of techniques for detrending include (but are not limited to) differencing each cardiac cycle with the prior cardiac cycle, and fitting and utilizing a regression model.

Provided in FIG. 6B is an example of the computed oxygen saturation overtime in which the second plethysmogram signal 603 of FIG. 6A was detrended. The computed oxygen saturation without detrending is shown in the darker data 611 and the computed oxygen saturation with detrending is shown in the light data 613.

In some embodiments, detrending may be achieved in part by determining a plurality of setpoints. The plurality of setpoints can be used to recalibrate the system. For example, the system can transmit light at a plurality of wavelengths each during a plurality of time windows. For each time window, the light can be sensed and converted to corresponding one or more plethysmogram signals. The one or more plethysmogram signals can be used to generate corresponding plethysmograms. Based on the plethysmogram(s), the system can generate a first plethysmogram setpoint. By identifying a difference between plethysmograms over time (e.g., between the first and second time windows), the system can calculate or otherwise determine a trend between the plethysmogram setpoints. The trend can be used to calibrate the system's determination of arterial oxygen saturation.

Method 500 also optionally corrects (507) a ratio of an unloaded cardiac cycle for variations of cuff pressure. To keep an artery in an unloaded state such that it has a constant volume, the pressure provided by the pressurized bladder of the cuff varies in accordance with the pressures the cardiac cycle. Further, overtime, a patient's blood pressure can change rapidly. Greater amounts of pressure on a body appendage can affect the DC component due to changes in light path. This affect is greater when arterial oxygen saturation is below healthy levels.

To correct for the effects of pressure changes on body appendage, a formula can be fitted to data comparing the effect of cuff pressure on the change of arterial oxygen saturation. From the formula, a correction factor can be implemented to adjust the ratio R of a cardiac cycle.

Method 500 further determines arterial oxygen saturation of an unloaded cardiac cycle using a calibrated ratio R utilizing a calibration curve. In some implementations, the calibrated ratio R is further corrected for trending plethysmogram signal and/or for variation in cuff pressure. In many implementations, ratio R is repeatedly recalibrated (and optionally re-corrected) and thus the latest computed calibration factor (and optionally the latest computed correction factors) are utilized to determine arterial oxygen saturation. The calibrated (and corrected) ratio R can be and a calibration curve can be utilized to determined arterial oxygen saturation.

The arterial oxygen saturation can be computed and updated on a continuous bases, such that arterial oxygen saturation is determined and updated for each cardiac cycle. When utilized in conjunction with a hemodynamic monitor, health monitor, or other monitoring device, the arterial oxygen saturation can be continuously displayed.

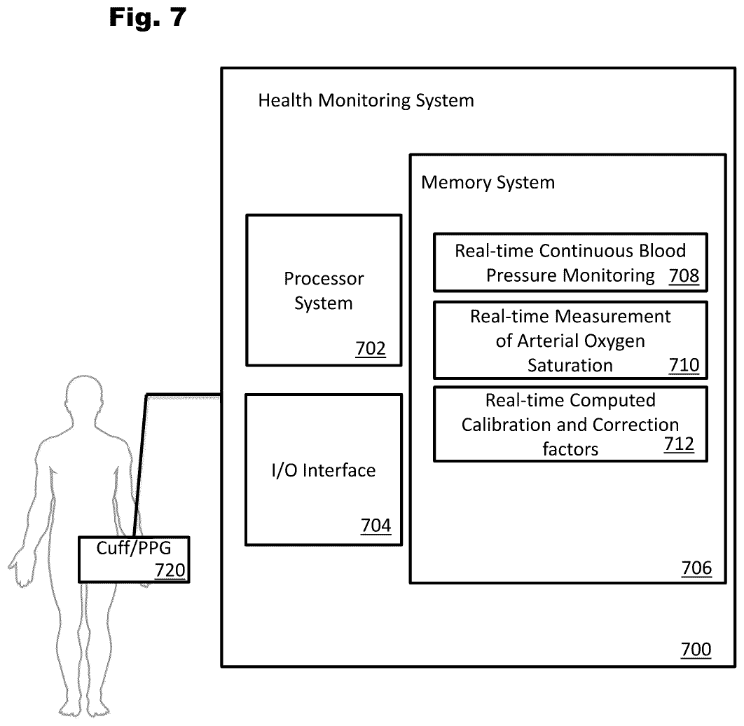

The systems and methods of the current disclosure can be utilized within a health monitoring system (e.g., hemodynamic monitoring system). Generally, the health monitoring system includes a blood pressure cuff comprising a pressurizable bladder and a PPG comprising an emitter capable of emitting at least two discrete wavelengths (or at least two discrete wavelength bands). Provided in FIG. 7 is an example of a health monitoring system 700 as would be utilized to monitor blood pressure and continuously (or continually) measure arterial oxygen saturation of an individual. Fitted upon a body appendage of the patient is the blood pressure cuff comprising the pressurizable bladder and the PPG 720 that can be in connection with a pneumatic system for providing fluidic pressure and further in connection health monitoring system 700 for performing real-time continuous blood pressure monitoring and real-time continuous (or continual) measurement of arterial oxygen saturation.

Health monitoring system 700 can comprise a computational system that comprises a processor system 702 and I/O interface 704 for input and output of data, such as data communicated between health monitoring system 700, blood pressure cuff and PPG 720, and a user interface. As can readily be appreciated, the processor system 702, I/O interface 704, and memory system 706 can be implemented using any of a variety of components appropriate to the requirements of specific applications including (but not limited to) CPUs, GPUs, ISPs, DSPs, wireless modems (e.g., Wi-Fi, Bluetooth modems), serial interfaces, volatile memory (e.g., DRAM) and/or non-volatile memory (e.g., SRAM, and/or NAND Flash).

Memory system 706 is capable of storing various data, applications, and models. It is to be understood that the listed data, applications and models are a representative sample of what can be stored in memory and that various memory systems may store some or all of the various data, applications, and models listed. Further, any combination of data, applications, and models can be stored, and in some implementations, various data, applications, and/or models are stored temporarily.

Health monitoring system 700 can utilize a number of applications stored within memory system 706 to be executed by processor system 702 to perform a set of instructions the direct the execution of various computational methods as described herein. Applications that can be stored within a memory system 706 include real-time continuous blood pressure monitoring 708 and real-time measurement of arterial oxygen saturation 710. Memory system 706 can further store real-time computed calibration and correction factors 712 that can be utilized by the real-time measurement of arterial oxygen saturation 710 application. The various applications can be provided as individual processes or as an ensemble of processes, each of which may be utilized to provide concurrent real-time continuous blood pressure monitoring and real-time measurement of arterial oxygen saturation. Real-time blood pressure and arterial oxygen saturation data can also optionally be stored on memory system 706 and/or displayed on a display screen via the I/O interface 704.

### EXAMPLES

### SET 1

Example 1. A method for continuous measurement of arterial oxygen saturation, comprising: transmitting a first wavelength of light and a second wavelength of light through a body appendage via an emitter within a blood pressure cuff; wherein the first wavelength of light and the second wavelength of light are discrete; sensing light signals of the first wavelength of light and of the second wavelength of light via a light sensor within the blood pressure cuff; receiving, using a health monitoring system, the light signals of the first wavelength of light and of the second wavelength of light, wherein the health monitoring system is in connection with the blood pressure cuff, wherein the light signals provide a plethysmogram for each of the first wavelength of light and of the second wavelength of light; computing, using the health monitoring system, a ratio between the plethysmogram of the first wavelength of light and the plethysmogram of the second wavelength of light; calibrating, using the health monitoring system, the computed ratio using a calibration factor; and computing, using the health monitoring system, arterial oxygen saturation using the calibrated and computed ratio.

Example 2. The method of example 1, wherein the light signals of the first wavelength are used to monitor blood pressure via a volume clamp method.

Example 3. The method of example 1 or 2, wherein transmitting a first wavelength of light and a second wavelength of light through a body appendage and sensing light signals of the first wavelength of light and of the second wavelength of light via a light sensor are performed while an artery within the body appendage is in an unloaded state.

Example 4. The method of example 3, wherein the ratio between the plethysmogram of the first wavelength of light and the plethysmogram of the second wavelength of light is computed using: wherein is ratio between the plethysmogram of the first wavelength of light and the plethysmogram of the second wavelength computed in an unloaded state; wherein represents an AC component of the first wavelength when the artery is the unloaded state; represents an AC component of the second wavelength when the artery is the unloaded state; is a DC component of the first wavelength when the artery is the unloaded state; and is a DC component of the second wavelength when the artery is the unloaded state.

Example 5. The method of example 3 or 4, wherein the blood pressure cuff comprises an inflatable bladder; wherein the inflatable bladder applies a pressure onto the body appendage such that the artery therein is in the unloaded state.

Example 6. The method of any one of examples 1-5, wherein the calibration factor is based on a ratio between the plethysmogram of the first wavelength of light and the plethysmogram of the second wavelength of light that is computed while an artery in the body appendage is a loaded state.

Example 7. The method of example 6, wherein the ratio between the plethysmogram of the first wavelength of light and the plethysmogram of the second wavelength of light is computed using: wherein is the ratio between the plethysmogram of the first wavelength of light and the plethysmogram of the second wavelength; wherein is an AC component of the first wavelength in the loaded state; is an AC component of the second wavelength in the loaded state; is a DC component of the first wavelength in the loaded state; and is a DC component of the second wavelength in the loaded state.

Example 8. The method of example 6 or 7, wherein a calibration is performed for determining a plethysmogram setpoint for blood pressure monitoring while the artery in the body appendage is the loaded state.

Example 9. The method of any one of examples 6-8, wherein the calibration factor is computed using: wherein is ratio between the plethysmogram of the first wavelength of light and the plethysmogram of the second wavelength computed in a loaded state and wherein is ratio between the plethysmogram of the first wavelength of light and the plethysmogram of the second wavelength computed in an unloaded state.

Example 10. The method of example 9, wherein is an averaged ratio of two more cardiac cycles.

Example 11. The method of example 9 or 10, wherein is an averaged ratio of two more cardiac cycles.

Example 12. The method of example 11, wherein the two more cardiac cycles comprise at least one cycle prior to a loaded cardiac cycle and at least one cycle subsequent to the loaded cardiac cycle; wherein is determined utilizing the loaded cardiac cycle.

Example 13. The method of example 12, wherein the loaded cardiac cycle is utilized to recalibrate a plethysmogram setpoint for blood pressure monitoring.

Example 14. The method of any one of examples 1-13 further comprising correcting, using the health monitoring system, the computed ratio for trending plethysmogram signal of the second wavelength of light when the plethysmogram signal of the second wavelength light exhibits trending.

Example 15. The method of any one of examples 1-14 further comprising correcting, using the health monitoring system, the computed ratio for variations of pressure provided by the blood pressure cuff.

Example 16. The method of any one of examples 1-15, wherein the first wavelength of light is infrared and the second wavelength of light is red.

Example 17. The method of any one of examples 1-16, wherein the body appendage is: an arm, a finger, a thumb, a wrist, an ankle, a leg, a toe, an ear, or a temple.

Example 18. The method of any one of examples 1-17 further comprising displaying the arterial oxygen saturation on a display is connection with the health monitoring system.

Example 19. A health monitoring system for continuous arterial oxygen saturation measurement, comprising: a blood pressure cuff in connection with a computational system, wherein the blood pressure cuff is configured to be fitted onto a body appendage; wherein the blood pressure cuff comprises an inflatable bladder, a light emitter, and a light sensor; wherein the light emitter is configured to transmit a first wavelength of light and a second wavelength of light through the body appendage; wherein the light sensor is configured to sense light signals of the first wavelength of light and of the second wavelength of light; wherein the computational system comprises a processor and memory; wherein the memory comprises one or more applications that comprise a set of instructions that are configured to direct the processor to: direct the light emitter to transmit the first wavelength of light and the second wavelength of light through a body appendage; direct the light sensor to sense light signals of the first wavelength of light and of the second wavelength of light; receive the light signals of the first wavelength of light and of the second wavelength of light, wherein the light signals provide a plethysmogram for each of the first wavelength of light and of the second wavelength of light; compute a ratio between the plethysmogram of the first wavelength of light and the plethysmogram of the second wavelength of light; calibrate the computed ratio using a calibration factor; and compute arterial oxygen saturation using the calibrated and computed ratio.

Example 20. The health monitoring system of example 19, wherein the light signals of the first wavelength are also used to monitor blood pressure via a volume clamp method.

Example 21. The health monitoring system of example 19 or 20, wherein the instructions that are configured to direct the processor to transmit a first wavelength of light and a second wavelength of light through a body appendage and that sense light signals of the first wavelength of light and of the second wavelength of light via a light sensor are configured to be performed while an artery within the body appendage is in an unloaded state.

Example 22. The health monitoring system of example 21, wherein the ratio between the plethysmogram of the first wavelength of light and the plethysmogram of the second wavelength of light is computed using: wherein is ratio between the plethysmogram of the first wavelength of light and the plethysmogram of the second wavelength computed in an unloaded state; wherein represents an AC component of the first wavelength when the artery is the unloaded state; represents an AC component of the second wavelength when the artery is the unloaded state; is a DC component of the first wavelength when the artery is the unloaded state; and is a DC component of the second wavelength when the artery is the unloaded state.

Example 23. The health monitoring system of example 21 or 22, wherein the blood pressure cuff comprises an inflatable bladder; wherein the inflatable bladder is configured to apply a pressure onto the body appendage such that the artery therein is in the unloaded state.

Example 24. The health monitoring system of any one of examples 19-23, wherein the calibration factor is based on a ratio between the plethysmogram of the first wavelength of light and the plethysmogram of the second wavelength of light that is computed while an artery in the body appendage is a loaded state.

Example 25. The health monitoring system of example 24, wherein the ratio between the plethysmogram of the first wavelength of light and the plethysmogram of the second wavelength of light is computed using: wherein is ratio between the plethysmogram of the first wavelength of light and the plethysmogram of the second wavelength; wherein is an AC component of the first wavelength; is an AC component of the second wavelength; is a DC component of the first wavelength; and is a DC component of the second wavelength.

Example 26. The health monitoring system of example 24 or 25, wherein the set of instructions is configured to direct the processor to direct the health monitoring system to perform a calibration for determining a plethysmogram setpoint for blood pressure monitoring while the artery in the body appendage is the loaded state.

Example 27. The health monitoring system of any one of examples 24-26, wherein the calibration factor is computed using: where wherein is ratio between the plethysmogram of the first wavelength of light and the plethysmogram of the second wavelength computed in a loaded state and wherein is ratio between the plethysmogram of the first wavelength of light and the plethysmogram of the second wavelength computed in an unloaded state.

Example 28. The health monitoring system of example 27, wherein is an averaged ratio of two more cardiac cycles.

Example 29. The health monitoring system of example 27 or 28, wherein is an averaged ratio of two more cardiac cycles.

Example 30. The health monitoring system of example 29, wherein the two more cardiac cycles comprise at least one cycle prior to a loaded cardiac cycle and at least one cycle subsequent to the loaded cardiac cycle; wherein is determined utilizing the loaded cardiac cycle.

Example 31. The health monitoring system of example 30, wherein the loaded cardiac cycle is utilized to recalibrate a plethysmogram setpoint for blood pressure monitoring.

Example 32. The health monitoring system of any one of examples 19-31, wherein the set of instructions that are further configured to direct the processor to correct the computed ratio for trending plethysmogram signal of the second wavelength of light when the plethysmogram signal of the second wavelength light exhibits trending.

Example 33. The health monitoring system of any one of examples 19-32, wherein the set of instructions that are further configured to direct the processor to correct the computed ratio for variations of pressure provided by the blood pressure cuff.

Example 34. The health monitoring system of any one of examples 19-33, wherein the first wavelength of light is infrared and the second wavelength of light is red.

Example 35. The health monitoring system of any one of examples 19-34, wherein the body appendage is: an arm, a finger, a thumb, a wrist, an ankle, a leg, a toe, an ear, or a temple.

Example 36. The health monitoring system of any one of examples 19-35 further comprising a display, wherein the set of instructions that are further configured to direct the processor to display the arterial oxygen saturation.

Example 37. A method of operating a noninvasive blood characteristic sensing system including a light emitter, a light sensor, and a inflatable bladder, the method comprising: (a) encircling a sensing region of a patient appendage with the inflatable bladder; (b) pressurizing the inflatable bladder to a constant pressurization during an open-loop calibration mode, comprising: (i) emitting light from the light emitter at a first wavelength into the sensing region of the patient appendage; (ii) emitting light from the light emitter at a second wavelength into the sensing region of the patient appendage; (iii) sensing light at the first wavelength from the light emitter at the light sensor, through the patient appendage; (iv) sensing light at the second wavelength from the light emitter at the light sensor, through the patient appendage; (v) generating a first sensed pleth signal based on the sensed light of the first wavelength; (vi) generating a second sensed pleth signal based on the sensed light of the second wavelength; (vii) generating a pleth setpoint based on the first or second sensed pleth signals, wherein the pleth setpoint corresponds to a resting, unstressed arterial volume; (c) generating a closed-loop R for determining arterial oxygen saturation measurement, comprising: (i) deriving and based on the first sensed pleth signal; (ii) deriving and based on the second sensed pleth signal; (iii) deriving the open-loop R using the formula: open-loop; (d) modulating pressurization of the inflatable bladder in a closed-loop control mode to partially clamp arterial volume within the sensing region via a closed-loop control algorithm, comprising: (i) emitting light from the light emitter at the first wavelength into the sensing region of the patient appendage; (ii) sensing light at the first wavelength from the light emitter at the light sensor, through the patient appendage; (iii) generating a third sensed pleth signal based on the sensed light of the first wavelength; (iv) emitting light from the light emitter at the second wavelength into the sensing region of the patient appendage; (v) sensing light at the second wavelength from the light emitter at the light sensor, through the patient appendage; (vi) generating a fourth sensed pleth signal based on the sensed light of the second wavelength; (vii) comparing the third or fourth sensed pleth signals with the pleth setpoint to generate a closed-loop error signal; (viii) modulating pressurization of the inflatable bladder responsive to the closed-loop error signal; (e) generating a closed-loop R for determining arterial oxygen saturation measurement, comprising: (i) deriving and based on the third sensed pleth signal; (ii) deriving and based on the fourth sensed pleth signal; (iii) deriving the closed-loop R using: closed-loop; (f) generating a calibrated R based on closed-loop R and a calibration factor, comprising: (i) calculating the calibration factor using: wherein is a ratio between a plethysmogram of the first wavelength of light and the plethysmogram of the second wavelength computed during the open-loop calibration mode and wherein is ratio between the plethysmogram of the first wavelength of light and the plethysmogram of the second wavelength computed in the closed-loop control algorithm; and (g) computing arterial oxygen saturation using the calibrated R and a pulse oximetry calibration curve.

Example 38. The method of claim 37, wherein and/or are averaged ratios of two more cardiac cycles.

Example 39. The method of claim 38, wherein the two more cardiac cycles of comprise at least one cycle prior to a loaded cardiac cycle and at least one cycle subsequent to the loaded cardiac cycle.

Example 40. The method of claim 39, wherein is determined utilizing the loaded cardiac cycle.

Example 41. A health monitoring system for continuous arterial oxygen saturation measurement, comprising: a blood pressure cuff in connection with a computational system, wherein the blood pressure cuff is configured to be fitted onto a body appendage; wherein the blood pressure cuff comprises an inflatable bladder, a light emitter, and a light sensor; wherein the light emitter is configured to transmit a first wavelength of light and a second wavelength of light through the body appendage; wherein the light sensor is configured to sense light signals of the first wavelength of light and of the second wavelength of light; wherein the computational system comprises a processor and memory; wherein the memory comprises one or more applications that comprise a set of instructions that are configured to direct the processor to: (a) encircle a sensing region of a patient appendage with the inflatable bladder; (b) pressurize the inflatable bladder to a constant pressurization during an open-loop calibration mode, comprising: (i) emitting light from the light emitter at a first wavelength into the sensing region of the patient appendage; (ii) emitting light from the light emitter at a second wavelength into the sensing region of the patient appendage; (iii) sensing light at the first wavelength from the light emitter at the light sensor, through the patient appendage; (iv) sensing light at the second wavelength from the light emitter at the light sensor, through the patient appendage; (v) generating a first sensed pleth signal based on the sensed light of the first wavelength; (vi) generating a second sensed pleth signal based on the sensed light of the second wavelength; (vii) generating a pleth setpoint based on the first or second sensed pleth signals, wherein the pleth setpoint corresponds to a resting, unstressed arterial volume; (c) generate a closed-loop R for determining arterial oxygen saturation measurement, comprising: (i) deriving and based on the first sensed pleth signal; (ii) deriving and based on the second sensed pleth signal; (iii) deriving the open-loop R using the formula: open-loop; (d) modulate pressurization of the inflatable bladder in a closed-loop control mode to partially clamp arterial volume within the sensing region via a closed-loop control algorithm, comprising: (i) emitting light from the light emitter at the first wavelength into the sensing region of the patient appendage; (ii) sensing light at the first wavelength from the light emitter at the light sensor, through the patient appendage; (iii) generating a third sensed pleth signal based on the sensed light of the first wavelength; (iv) emitting light from the light emitter at the second wavelength into the sensing region of the patient appendage; (v) sensing light at the second wavelength from the light emitter at the light sensor, through the patient appendage; (vi) generating a fourth sensed pleth signal based on the sensed light of the second wavelength; (vii) comparing the third or fourth sensed pleth signals with the pleth setpoint to generate a closed-loop error signal; (viii) modulating pressurization of the inflatable bladder responsive to the closed-loop error signal; (e) generate a closed-loop R for determining arterial oxygen saturation measurement, comprising: (i) deriving and based on the third sensed pleth signal; (ii) deriving and based on the fourth sensed pleth signal; (iii) deriving the closed-loop R using: closed-loop; (f) generate a calibrated R based on closed-loop R and a calibration factor, comprising: (i) calculating the calibration factor using: wherein is a ratio between a plethysmogram of the first wavelength of light and the plethysmogram of the second wavelength computed during the open-loop calibration mode and wherein is ratio between the plethysmogram of the first wavelength of light and the plethysmogram of the second wavelength computed in the closed-loop control algorithm; and g) compute arterial oxygen saturation using the calibrated R and a pulse oximetry calibration curve.

Example 42. The health monitoring system of example 41, wherein and/or are averaged ratios of two more cardiac cycles.

Example 43. The health monitoring system of example 42, wherein the two more cardiac cycles of comprise at least one cycle prior to a loaded cardiac cycle and at least one cycle subsequent to the loaded cardiac cycle

Example 44. The health monitoring system of example 43, wherein is determined utilizing the loaded cardiac cycle.

### SET 1

In a 1st Example, a method for measurement of arterial oxygen saturation, comprising: transmitting, using at least one emitter of a blood pressure cuff, a first wavelength of light and a second wavelength of light through a body appendage; sensing, using a light sensor of the blood pressure cuff, light signals of the first wavelength of light and of the second wavelength of light; onverting, using a health monitoring system, the light signals of the first wavelength of light and of the second wavelength of light into corresponding plethysmogram signals, wherein the health monitoring system is in connection with the blood pressure cuff, wherein the plethysmogram signals are configured to provide a plethysmogram for each of the first wavelength of light and of the second wavelength of light; computing, using the health monitoring system, a ratio between the plethysmogram of the first wavelength of light and the plethysmogram of the second wavelength of light; calibrating, using the health monitoring system, the computed ratio using a calibration factor; and computing, using the health monitoring system, arterial oxygen saturation using the calibration factor and the computed ratio.

In a 2nd Example, the method of Example 1, wherein sensing the light signals of the first wavelength of light and of the second wavelength of light comprises applying, using the blood pressure cuff, a pressure to the body appendage such that the artery therein is in the unloaded state.

In a 3rd Example, the method of any of Examples 1-2, further comprising determining a blood pressure of the body appendage via a volume clamp method using the light signals of the first wavelength of light or of the second wavelength of light.

In a 4th Example, the method of any of Examples 1-3, wherein transmitting the first wavelength of light and the second wavelength of light through the body appendage and sensing light signals of the first wavelength of light and of the second wavelength of light are performed while an artery within the body appendage is in an unloaded state.

In a 5th Example, the method of Example 4, wherein the ratio between the plethysmogram of the first wavelength of light and the plethysmogram of the second wavelength of light is computed using:

$${R}_{Unl} = \frac{( \frac{Δ {d}_{Unl \_λ1}}{{DC}_{Unl \_λ1}} )}{( \frac{Δ {d}_{Unl \_λ2}}{{DC}_{Unl \_λ2}} )}$$

wherein R_{Unl} is the ratio between the plethysmogram of the first wavelength of light and the plethysmogram of the second wavelength when the artery is in the unloaded state; and wherein: Δd_{Unl_λ1} represents an AC component of the first wavelength when the artery is the unloaded state; Δd_{Unl_λ2} represents an AC component of the second wavelength when the artery is the unloaded state; DC_{Unl_λ1} is a DC component of the first wavelength when the artery is the unloaded state; and DC_{Unl_λ2} is a DC component of the second wavelength when the artery is the unloaded state.

In a 6th Example, the method of any of Examples 4-5, wherein the blood pressure cuff comprises an inflatable bladder and wherein the inflatable bladder applies a pressure onto the body appendage such that the artery therein is in the unloaded state.

In a 7th Example, the method of any one of Examples 1-5, wherein the calibration factor is based on a ratio between the plethysmogram of the first wavelength of light and the plethysmogram of the second wavelength of light for an artery in the body appendage is a loaded state.

In a 8th Example, the method of Example 7, wherein the ratio between the plethysmogram of the first wavelength of light and the plethysmogram of the second wavelength of light is computed using:

$$R = \frac{( \frac{{AC}_{λ1}}{{DC}_{λ1}} )}{( \frac{{AC}_{λ2}}{{DC}_{λ2}} )}$$

wherein R is the ratio between the plethysmogram of the first wavelength of light and the plethysmogram of the second wavelength; and wherein: AC_{λ1} is an AC component of the first wavelength in the loaded state; AC_{λ2} is an AC component of the second wavelength in the loaded state; DC_{λ1} is a DC component of the first wavelength in the loaded state; and DC_{λ2} is a DC component of the second wavelength in the loaded state.

In a 9th Example, the method of any of Examples 7-8, further comprising determining, using a second calibration factor, a plethysmogram setpoint for blood pressure monitoring while the artery in the body appendage is the loaded state.

In a 10th Example, the method of any one of Examples 7-9, wherein the calibration factor is computed using:

$$Cal\_factor = \frac{{R}_{Loa}}{{R}_{Unl}}$$

wherein R_{Loa} is a ratio between the plethysmogram of the first wavelength of light and the plethysmogram of the second wavelength in the loaded state, and wherein R_{Unl} is a ratio between the plethysmogram of the first wavelength of light and the plethysmogram of the second wavelength in the unloaded state.

In a 11th Example, the method of Example 10, wherein R_{Loa} is an ratio averaged over two or more cardiac cycles.

In a 12th Example, the method of any of Examples 10-11, wherein R_{Unl} is a ratio averaged over two or more cardiac cycles.

In a 13th Example, the method of Example 12, wherein the two or more cardiac cycles comprise at least one cycle prior to a loaded cardiac cycle and at least one cycle subsequent to the loaded cardiac cycle, and wherein R_{Loa} is determined based on the loaded cardiac cycle.

In a 14th Example, the method of Example 13, further comprising recalibrating, based on the loaded cardiac cycle, the plethysmogram setpoint for blood pressure monitoring.

In a 15th Example, the method of any one of Examples 1-14, further comprising correcting, using the health monitoring system, the computed ratio for a trending plethysmogram signal of the second wavelength of light when the plethysmogram signal of the second wavelength light exhibits trending.

In a 16th Example, the method of any one of Examples 1-15, further comprising correcting, using the health monitoring system, the computed ratio for variations of pressure applied by the blood pressure cuff.

In a 17th Example, the method of any one of Examples 1-16, wherein the first wavelength of light is red and the second wavelength of light is infrared.

In a 18th Example, the method of any one of Examples 1-17, wherein the body appendage comprises at least one of: an arm, a finger, a thumb, a wrist, an ankle, a leg, a toe, an ear, or a temple.

In a 19th Example, the method of any one of Examples 1-18, further comprising displaying the arterial oxygen saturation on a display in connection with the health monitoring system.

In a 20th Example, the method of any one of Examples 1-19, wherein transmitting the first wavelength of light and the second wavelength of light through the body appendage comprises transmitting the first wavelength of light and the second wavelength of light through a portion of the blood pressure cuff.

In a 21st Example, the method of Example 20, wherein the portion of the blood pressure cuff comprises an inflatable bladder.

In a 22nd Example, a health monitoring system for arterial oxygen saturation measurement, comprising: a blood pressure cuff in connection with a computational system, wherein the blood pressure cuff is configured to be fitted onto a body appendage, wherein the blood pressure cuff comprises: an inflatable bladder; a light emitter, wherein the light emitter is configured to transmit a first wavelength of light and a second wavelength of light through the body appendage; and a light sensor, wherein the light sensor is configured to sense light signals of the first wavelength of light and of the second wavelength of light; a hardware processor; and a non-transitory memory comprising a set of instructions, wherein the set of instructions, when executed by the processor, are configured to direct the processor to: direct the light emitter to transmit the first wavelength of light and the second wavelength of light through the body appendage; direct the light sensor to sense light signals of the first wavelength of light and of the second wavelength of light; convert the light signals of the first wavelength of light and of the second wavelength of light into corresponding plethysmogram signals, wherein the plethysmogram signals are configured to provide a plethysmogram for each of the first wavelength of light and of the second wavelength of light; compute a ratio between the plethysmogram of the first wavelength of light and the plethysmogram of the second wavelength of light; calibrate the computed ratio using a calibration factor; and compute arterial oxygen saturation using the calibration factor and the computed ratio.

In a 23rd Example, the health monitoring system of Example 22, wherein the light signals of the first or second wavelength are also used to monitor blood pressure via a volume clamp method.

In a 24th Example, the health monitoring system of any of Examples 22-23, wherein the instructions that are configured to direct the processor to transmit a first wavelength of light and a second wavelength of light through a body appendage and that sense light signals of the first wavelength of light and of the second wavelength of light via a light sensor are configured to be performed while an artery within the body appendage is in an unloaded state.

In a 25th Example, the health monitoring system of Example 24, wherein the ratio between the plethysmogram of the first wavelength of light and the plethysmogram of the second wavelength of light is computed using:

$${R}_{Unl} = \frac{( \frac{Δ {d}_{Unl \_λ1}}{{DC}_{Unl \_λ1}} )}{( \frac{Δ {d}_{Unl \_λ2}}{{DC}_{Unl \_λ2}} )}$$

wherein R_{Unl} is ratio between the plethysmogram of the first wavelength of light and the plethysmogram of the second wavelength computed in an unloaded state; wherein Δd_{Unl_λ1} represents an AC component of the first wavelength when the artery is the unloaded state; Δd_{Unl_λ2} represents an AC component of the second wavelength when the artery is the unloaded state; DC_{Unl_λ1} is a DC component of the first wavelength when the artery is the unloaded state; and DC_{Unl_λ2} is a DC component of the second wavelength when the artery is the unloaded state.

In a 26th Example, the health monitoring system of any of Examples 24-25, wherein the blood pressure cuff comprises an inflatable bladder; wherein the inflatable bladder is configured to apply a pressure onto the body appendage such that the artery therein is in the unloaded state.

In a 27th Example, the health monitoring system of any one of Examples 19-23, wherein the calibration factor is based on a ratio between the plethysmogram of the first wavelength of light and the plethysmogram of the second wavelength of light that is computed while an artery in the body appendage is a loaded state.

In a 28th Example, the health monitoring system of Example 27, wherein the ratio between the plethysmogram of the first wavelength of light and the plethysmogram of the second wavelength of light is computed using:

$$R = \frac{( \frac{{AC}_{λ1}}{{DC}_{λ1}} )}{( \frac{{AC}_{λ2}}{{DC}_{λ2}} )}$$

wherein R is ratio between the plethysmogram of the first wavelength of light and the plethysmogram of the second wavelength; wherein AC_{λ1} is an AC component of the first wavelength; AC_{λ2} is an AC component of the second wavelength; DC_{λ1} is a DC component of the first wavelength; and DC_{λ2} is a DC component of the second wavelength.

In a 29th Example, the health monitoring system of any of Examples 27-28, wherein the set of instructions is configured to direct the processor to direct the health monitoring system to perform a calibration for determining a plethysmogram setpoint for blood pressure monitoring while the artery in the body appendage is the loaded state.

In a 30th Example, the health monitoring system of any of Examples 27-29, wherein the calibration factor is computed using:

$$Cal\_factor = \frac{{R}_{Loa}}{{R}_{Unl}}$$

where wherein R_{Loa} is ratio between the plethysmogram of the first wavelength of light and the plethysmogram of the second wavelength computed in a loaded state and wherein R_{Unl} is ratio between the plethysmogram of the first wavelength of light and the plethysmogram of the second wavelength computed in an unloaded state.

In a 31st Example, the health monitoring system of Example 30, wherein R_{Loa} is an averaged ratio of two more cardiac cycles.

In a 32nd Example, the health monitoring system of Example 30 or 31, wherein R_{Unl} is an averaged ratio of two more cardiac cycles.

In a 33rd Example, the health monitoring system of Example 32, wherein the two more cardiac cycles comprise at least one cycle prior to a loaded cardiac cycle and at least one cycle subsequent to the loaded cardiac cycle; wherein R_{Loa} is determined utilizing the loaded cardiac cycle.

In a 34th Example, the health monitoring system of Example 33, wherein the loaded cardiac cycle is utilized to recalibrate a plethysmogram setpoint for blood pressure monitoring.

In a 35th Example, the health monitoring system of any one of Examples 22-34, wherein the set of instructions are further configured to direct the processor to correct the computed ratio for trending plethysmogram signal of the second wavelength of light when the plethysmogram signal of the second wavelength light exhibits trending.

In a 36th Example, the health monitoring system of any one of Examples 22-35, wherein the set of instructions are further configured to direct the processor to correct the computed ratio for variations of pressure provided by the blood pressure cuff.

In a 37th Example, the health monitoring system of any one of Examples 22-36, wherein the first wavelength of light is infrared and the second wavelength of light is red.

In a 38th Example, the health monitoring system of any one of Examples 22-37, wherein the body appendage is: an arm, a finger, a thumb, a wrist, an ankle, a leg, a toe, an ear, or a temple.

In a 39th Example, the health monitoring system of any one of Examples 22-38, further comprising a display, wherein the set of instructions are further configured to direct the processor to display the arterial oxygen saturation.

In a 40th Example, the health monitoring system of any one of Examples 22-39, wherein transmitting the first wavelength of light and the second wavelength of light through the body appendage comprises transmitting the first wavelength of light and the second wavelength of light through a portion of the blood pressure cuff.

In a 41st Example, the health monitoring system of Example 40, wherein the portion of the blood pressure cuff comprises an inflatable bladder.

In a 42nd Example, a method of operating a noninvasive blood characteristic sensing system including a light emitter, a light sensor, and an inflatable bladder, the method comprising: encircling a sensing region of a patient appendage with the inflatable bladder; pressurizing the inflatable bladder to a constant pressurization during an open-loop calibration mode, comprising: emitting light from the light emitter at a first wavelength into the sensing region of the patient appendage; emitting light from the light emitter at a second wavelength into the sensing region of the patient appendage; sensing light at the first wavelength from the light emitter at the light sensor, through the patient appendage; sensing light at the second wavelength from the light emitter at the light sensor, through the patient appendage; generating a first sensed pleth signal based on the sensed light of the first wavelength; generating a second sensed pleth signal based on the sensed light of the second wavelength; generating a pleth setpoint based on the first or second sensed pleth signals, wherein the pleth setpoint corresponds to an unstressed arterial volume; generating a open-loop R for determining arterial oxygen saturation measurement, comprising: deriving AC_{λ1} and DC_{λ1} based on the first sensed pleth signal; deriving AC_{λ2} and DC_{λ2} based on the second sensed pleth signal; and deriving the open-loop R using the formula:

$$open - loop R = \frac{( \frac{{AC}_{λ1}}{{DC}_{λ1}} )}{( \frac{{AC}_{λ2}}{{DC}_{λ2}} )}$$

modulating pressurization of the inflatable bladder in a closed-loop control mode to at least partially clamp arterial volume within the sensing region via a closed-loop control algorithm, comprising: emitting light from the light emitter at the first wavelength into the sensing region of the patient appendage; sensing light at the first wavelength from the light emitter at the light sensor, through the patient appendage; generating a third sensed pleth signal based on the sensed light of the first wavelength; emitting light from the light emitter at the second wavelength into the sensing region of the patient appendage; sensing light at the second wavelength from the light emitter at the light sensor, through the patient appendage; generating a fourth sensed pleth signal based on the sensed light of the second wavelength; comparing the third or fourth sensed pleth signals with the pleth setpoint to generate a closed-loop error signal; and modulating pressurization of the inflatable bladder responsive to the closed-loop error signal; generating a closed-loop R for determining arterial oxygen saturation measurement, comprising: deriving AC_{Unl_λ1} and DC_{Unl_λ1} based on the third sensed pleth signal; deriving AC_{Unl_λ2} and DC_{Unl_λ2} based on the fourth sensed pleth signal; deriving the closed-loop R using the formula:

$$closed - loop R = \frac{( \frac{{AC}_{Unl \_λ1}}{{DC}_{Unl \_λ1}} )}{( \frac{{AC}_{Unl \_λ2}}{{DC}_{Unl \_λ2}} )}$$

generating a calibrated R based on closed-loop R and a calibration factor; and computing an arterial oxygen saturation using the calibrated R and a pulse oximetry calibration curve.

In a 43rd Example, the method of Example 42, wherein the calibration factor is based on an R value from pleth signals for both the first and second wavelengths measured during the open-loop calibration mode and during the closed-loop calibration mode.

In a 44th Example, the method of Example 43, wherein generating the calibrated R comprises calculating the calibration factor using the following equation:

$$calibration factor = \frac{{R}_{Loa}}{{R}_{Unl}}$$

wherein R_{Loa} is a ratio between the pleth signal of the first wavelength of light and the pleth signal of the second wavelength of the open-loop calibration mode and wherein R_{Unl} is ratio between the pleth signal of the first wavelength of light and the pleth signal of the second wavelength of the closed-loop control algorithm.

In a 45th Example, the method of Example 44, wherein R_{Loa} and/or R_{Unl} are averaged ratios of two more cardiac cycles.

In a 46th Example, the method of Example 45, wherein the two more cardiac cycles of R_{Unl} comprise at least one cycle prior to a loaded cardiac cycle used to calculate R_{Loa} and at least one cycle subsequent to the loaded cardiac cycle.

In a 47th Example, a health monitoring system for arterial oxygen saturation measurement, comprising: a blood pressure cuff in connection with a computational system, wherein the blood pressure cuff is configured to be fitted onto a body appendage; wherein the blood pressure cuff comprises: an inflatable bladder, a light emitter, wherein the light emitter is configured to transmit a first wavelength of light and a second wavelength of light through the body appendage; and a light sensor, wherein the light sensor is configured to sense light signals of the first wavelength of light and of the second wavelength of light; a processor; and a memory, wherein the memory comprises one or more applications that comprise a set of instructions that are configured to direct the processor to perform the method of any of Examples 42-47.

In a 48th Example, a health monitoring system for arterial oxygen saturation measurement, comprising: a blood pressure cuff in connection with a computational system, wherein the blood pressure cuff is configured to be fitted onto a body appendage, wherein the blood pressure cuff comprises: an inflatable bladder; a light emitter comprising an LED, wherein the LED is configured to transmit a first wavelength of light and a second wavelength of light through the body appendage; and a light sensor, wherein the light sensor is configured to sense light signals of the first wavelength of light and of the second wavelength of light; a hardware processor; and a non-transitory memory comprising a set of instructions, wherein the set of instructions, when executed by the processor, are configured to direct the processor to: direct the LED to transmit the first wavelength of light and the second wavelength of light through the body appendage; direct the light sensor to sense light signals of the first wavelength of light and of the second wavelength of light; convert the light signals of the first wavelength of light and of the second wavelength of light into corresponding plethysmogram signals, wherein the plethysmogram signals are configured to provide a plethysmogram for each of the first wavelength of light and of the second wavelength of light; compute a ratio between the plethysmogram of the first wavelength of light and the plethysmogram of the second wavelength of light; and compute arterial oxygen saturation using the computed ratio.

In a 49th Example, the health monitoring system of Example 48, wherein directing the LED to transmit the first wavelength of light and the second wavelength of light through the body appendage comprises directing the LED to transmit the first wavelength of light and the second wavelength of light through a portion of the blood pressure cuff.

In a 50th Example, the health monitoring system of Example 49, wherein the portion of the blood pressure cuff comprises an inflatable bladder.

In a 51st Example, the health monitoring system of any of Examples 48-50, wherein the light signals of the second wavelength are also used to monitor blood pressure via a volume clamp method.

In a 52nd Example, the health monitoring system of Example 48 or 51, wherein the instructions that are configured to direct the processor to transmit a first wavelength of light and a second wavelength of light through a body appendage and that sense light signals of the first wavelength of light and of the second wavelength of light via a light sensor are configured to be performed while an artery within the body appendage is in an unloaded state.

In a 53rd Example, the health monitoring system of Example 52, wherein the ratio between the plethysmogram of the first wavelength of light and the plethysmogram of the second wavelength of light is computed using:

$${R}_{Unl} = \frac{( \frac{Δ {d}_{Unl\_ λ 1}}{{DC}_{Unl\_ λ1}} )}{( \frac{Δ {d}_{Unl\_ λ 2}}{{DC}_{Unl\_ λ 2}} )}$$

wherein R_{Unl} is ratio between the plethysmogram of the first wavelength of light and the plethysmogram of the second wavelength computed in an unloaded state; wherein Δd_{Unl_λ1} represents an AC component of the first wavelength when the artery is the unloaded state; Δd_{Unl_λ2} represents an AC component of the second wavelength when the artery is the unloaded state; DC_{Unl_λ1} is a DC component of the first wavelength when the artery is the unloaded state; and DC_{Unl_λ2} is a DC component of the second wavelength when the artery is the unloaded state.

In a 54th Example, the health monitoring system of Examples 52-53, wherein the blood pressure cuff comprises an inflatable bladder; wherein the inflatable bladder is configured to apply a pressure onto the body appendage such that the artery therein is in the unloaded state.

In a 55th Example, the health monitoring system of any one of Examples 48-54, wherein the calibration factor is based on a ratio between the plethysmogram of the first wavelength of light and the plethysmogram of the second wavelength of light that is computed while an artery in the body appendage is a loaded state.

In a 56th Example, the health monitoring system of Example 55, wherein the ratio between the plethysmogram of the first wavelength of light and the plethysmogram of the second wavelength of light is computed using:

$$R = \frac{( \frac{{AC}_{λ1}}{{DC}_{λ1}} )}{( \frac{{AC}_{λ 2}}{{DC}_{λ 2}} )}$$

wherein R is ratio between the plethysmogram of the first wavelength of light and the plethysmogram of the second wavelength; wherein AC_{λ1} is an AC component of the first wavelength; AC_{λ2} is an AC component of the second wavelength; DC_{λ1} is a DC component of the first wavelength; and DC_{λ2} is a DC component of the second wavelength.

In a 57th Example, the health monitoring system of Example 55 or 56, wherein the set of instructions is configured to direct the processor to direct the health monitoring system to perform a calibration for determining a plethysmogram setpoint for blood pressure monitoring while the artery in the body appendage is the loaded state.

In a 58th Example, the health monitoring system of any one of Examples 55-57, wherein the calibration factor is computed using:

$$Cal\_factor = \frac{{R}_{Loa}}{{R}_{Unl}}$$

where wherein R_{Loa} is ratio between the plethysmogram of the first wavelength of light and the plethysmogram of the second wavelength computed in a loaded state and wherein R_{Unl} is ratio between the plethysmogram of the first wavelength of light and the plethysmogram of the second wavelength computed in an unloaded state.

In a 59th Example, the health monitoring system of Example 58, wherein R_{Loa} is an averaged ratio of two more cardiac cycles.

In a 60th Example, the health monitoring system of Example 58 or 59, wherein R_{Unl} is an averaged ratio of two more cardiac cycles.

In a 61st Example, the health monitoring system of Example 60, wherein the two more cardiac cycles comprise at least one cycle prior to a loaded cardiac cycle and at least one cycle subsequent to the loaded cardiac cycle; wherein R_{Loa} is determined utilizing the loaded cardiac cycle.

In a 62nd Example, the health monitoring system of Example 61, wherein the loaded cardiac cycle is utilized to recalibrate a plethysmogram setpoint for blood pressure monitoring.

In a 63rd Example, the health monitoring system of any one of Examples 48-62 wherein the set of instructions are further configured to direct the processor to correct the computed ratio for trending plethysmogram signal of the second wavelength of light when the plethysmogram signal of the second wavelength light exhibits trending.

In a 64th Example, the health monitoring system of any one of Examples 48-63, wherein the set of instructions are further configured to direct the processor to correct the computed ratio for variations of pressure provided by the blood pressure cuff.

In a 65th Example, the health monitoring system of any one of Examples 48-64, wherein the first wavelength of light is infrared and the second wavelength of light is red.

In a 66th Example, the health monitoring system of any one of Examples 48-65, wherein the body appendage is: an arm, a finger, a thumb, a wrist, an ankle, a leg, a toe, an ear, or a temple.

In a 67th Example, the health monitoring system of any one of Examples 48-66 further comprising a display, wherein the set of instructions are further configured to direct the processor to display the arterial oxygen saturation.

In a 68th Example, a method for arterial oxygen saturation measurement, comprising: transmitting, using an LED of an emitter, the first wavelength of light and the second wavelength of light through the body appendage; direct the light sensor to sense light signals of the first wavelength of light and of the second wavelength of light; convert the light signals of the first wavelength of light and of the second wavelength of light into corresponding plethysmogram signals, wherein the plethysmogram signals are configured to provide a plethysmogram for each of the first wavelength of light and of the second wavelength of light; compute a ratio between the plethysmogram of the first wavelength of light and the plethysmogram of the second wavelength of light; and compute arterial oxygen saturation using the computed ratio.

In a 69th Example, the method of Example 68, wherein sensing the light signals of the first wavelength of light and of the second wavelength of light comprises applying, using the blood pressure cuff, a pressure to the body appendage such that the artery therein is in the unloaded state.

In a 70th Example, the method of any of Examples 68-69, further comprising determining, based on the plethysmogram signals of the first wavelength, a blood pressure of the body appendage.

In a 71st Example, the method of any of Examples 68-70, wherein transmitting the first wavelength of light and the second wavelength of light through the body appendage and sensing light signals of the first wavelength of light and of the second wavelength of light are performed while an artery within the body appendage is in an unloaded state.

In a 72nd Example, the method of Example 71, wherein the ratio between the plethysmogram of the first wavelength of light and the plethysmogram of the second wavelength of light is computed using:

$${R}_{Unl} = \frac{( \frac{Δ {d}_{Unl\_ λ 1}}{{DC}_{Unl\_ λ1}} )}{( \frac{Δ {d}_{Unl\_ λ 2}}{{DC}_{Unl\_ λ 2}} )}$$

wherein R_{Unl} is the ratio between the plethysmogram of the first wavelength of light and the plethysmogram of the second wavelength when the artery is in the unloaded state; and wherein: Δd_{Unl_λ1} represents an AC component of the first wavelength when the artery is the unloaded state; Δd_{Unl_λ2} represents an AC component of the second wavelength when the artery is the unloaded state; DC_{Unl_λ1} is a DC component of the first wavelength when the artery is the unloaded state; and DC_{Unl_λ2} is a DC component of the second wavelength when the artery is the unloaded state.

In a 73rd Example, the method of any of Examples 71-72, wherein the blood pressure cuff comprises an inflatable bladder and wherein the inflatable bladder applies a pressure onto the body appendage such that the artery therein is in the unloaded state.

In a 74th Example, the method of any one of Examples 68-73, wherein the calibration factor is based on a ratio between the plethysmogram of the first wavelength of light and the plethysmogram of the second wavelength of light for an artery in the body appendage is a loaded state.

In a 75th Example, the method of Example 74, wherein the ratio between the plethysmogram of the first wavelength of light and the plethysmogram of the second wavelength of light is computed using:

$$R = \frac{( \frac{{AC}_{λ1}}{{DC}_{λ1}} )}{( \frac{{AC}_{λ 2}}{{DC}_{λ 2}} )}$$

wherein R is the ratio between the plethysmogram of the first wavelength of light and the plethysmogram of the second wavelength; and wherein: AC_{λ1} is an AC component of the first wavelength in the loaded state; AC_{λ2} is an AC component of the second wavelength in the loaded state; DC_{λ1} is a DC component of the first wavelength in the loaded state; and DC_{λ2} is a DC component of the second wavelength in the loaded state.

In a 76th Example, the method of any of Examples 74-75, further comprising determining, using a second calibration factor, a plethysmogram setpoint for blood pressure monitoring while the artery in the body appendage is the loaded state.

In a 77th Example, the method of any one of Examples 74-76, wherein the calibration factor is computed using:

$$Cal\_factor = \frac{{R}_{Loa}}{{R}_{Unl}}$$

wherein R_{Loa} is a ratio between the plethysmogram of the first wavelength of light and the plethysmogram of the second wavelength in the loaded state, and wherein R_{Unl} is a ratio between the plethysmogram of the first wavelength of light and the plethysmogram of the second wavelength in the unloaded state.

In a 78th Example, the method of Example 77, wherein R_{Loa} is an ratio averaged over two or more cardiac cycles.

In a 79th Example, the method of any of Examples 77-78, wherein R_{Unl} is a ratio averaged over two or more cardiac cycles.

In a 80th Example, the method of Example 79, wherein the two or more cardiac cycles comprise at least one cycle prior to a loaded cardiac cycle and at least one cycle subsequent to the loaded cardiac cycle, and wherein R_{Loa} is determined based on the loaded cardiac cycle.

In an 81st Example, the method of Example 80, further comprising recalibrating, based on the loaded cardiac cycle, the plethysmogram setpoint for blood pressure monitoring.

In an 82nd Example, the method of any one of Examples 68-81, further comprising correcting, using the health monitoring system, the computed ratio for a trending plethysmogram signal of the second wavelength of light when the plethysmogram signal of the second wavelength light exhibits trending.

In an 83rd Example, the method of any one of Examples 68-82, further comprising correcting, using the health monitoring system, the computed ratio for variations of pressure applied by the blood pressure cuff.

In an 84th Example, the method of any one of Examples 68-83, wherein the first wavelength of light is infrared and the second wavelength of light is red.

In an 85th Example, the method of any one of Examples 68-84, wherein the body appendage comprises at least one of: an arm, a finger, a thumb, a wrist, an ankle, a leg, a toe, an ear, or a temple.

In an 86th Example, the method of any one of Examples 68-85, further comprising displaying the arterial oxygen saturation on a display in connection with the health monitoring system.

In an 87th Example, the method of any one of Examples 68-86, wherein transmitting the first wavelength of light and the second wavelength of light through the body appendage comprises transmitting the first wavelength of light and the second wavelength of light through a portion of the blood pressure cuff.

In an 88th Example, the method of Example 87, wherein the portion of the blood pressure cuff comprises an inflatable bladder.

In an 89th Example, a health monitoring system for arterial oxygen saturation measurement, comprising: a blood pressure cuff in connection with a computational system, wherein the blood pressure cuff is configured to be fitted onto a body appendage, wherein the blood pressure cuff comprises: an inflatable bladder; at least one light emitter configured to transmit a first wavelength of light and a second wavelength of light through the body appendage; and a light sensor configured to sense light signals of the first wavelength of light and of the second wavelength of light; a hardware processor; and a non-transitory memory comprising a set of instructions, wherein the set of instructions, when executed by the processor, are configured to direct the processor to: direct the inflatable bladder to apply a constant pressurization to the body appendage; direct the light emitter to transmit, during the constant pressurization, the first wavelength of light and the second wavelength of light through the body appendage; direct the light sensor to sense light signals of the first wavelength of light and of the second wavelength of light; convert the light signals of the first wavelength of light and of the second wavelength of light into corresponding plethysmogram signals, wherein the plethysmogram signals are configured to provide a plethysmogram for each of the first wavelength of light and of the second wavelength of light; compute a ratio between the plethysmogram of the first wavelength of light and the plethysmogram of the second wavelength of light; and compute arterial oxygen saturation using the computed ratio.

In a 90th Example, the health monitoring system of Example 89, wherein applying the constant pressurization to the body appendage comprises applying the constant pressurization for at least a full cardiac cycle.

In a 91st Example, the health monitoring system of any of Examples 89-90, wherein applying the constant pressurization to the body appendage comprises applying a pressure no greater than an ambient pressure.

In a 92nd Example, a health monitoring system for arterial oxygen saturation measurement, comprising: a blood pressure cuff in connection with a computational system, wherein the blood pressure cuff is configured to be fitted onto a body appendage, wherein the blood pressure cuff comprises: an inflatable bladder; a light emitter configured to transmit, through the body appendage, a first wavelength of light at a first time and a second wavelength of light at a second time; and a light sensor configured to sense light signals of the first wavelength of light and of the second wavelength of light; a hardware processor; and a non-transitory memory comprising a set of instructions, wherein the set of instructions, when executed by the processor, are configured to direct the processor to: direct the light emitter to transmit, through the body appendage, the first wavelength of light at the first time and the second wavelength of light at the second time; direct the light sensor to sense light signals of the first wavelength of light and of the second wavelength of light; convert the light signals of the first wavelength of light and of the second wavelength of light into corresponding plethysmogram signals, wherein the plethysmogram signals are configured to provide a plethysmogram for each of the first wavelength of light and of the second wavelength of light; compute a ratio between the plethysmogram of the first wavelength of light and the plethysmogram of the second wavelength of light; and compute arterial oxygen saturation using the calibration factor and the computed ratio.

In a 93rd Example, the health monitoring system of Example 92, wherein a difference between the second time and the first time is less than a full cardiac cycle.

In a 94th Example, a health monitoring system for arterial oxygen saturation measurement, comprising: a blood pressure cuff in connection with a computational system, wherein the blood pressure cuff is configured to be fitted onto a body appendage, wherein the blood pressure cuff comprises: an inflatable bladder; at least one light emitter configured to transmit, through the body appendage, a first wavelength of light and a second wavelength of light; and a light sensor configured to sense light signals of the first wavelength of light and of the second wavelength of light; a hardware processor; and a non-transitory memory comprising a set of instructions, wherein the set of instructions, when executed by the processor, are configured to direct the processor to: direct, during a first time window, the light emitter to transmit, through the body appendage, the first wavelength of light and the second wavelength of light; direct the light sensor to sense light signals of the first wavelength of light and of the second wavelength of light; convert the light signals of the first wavelength of light and of the second wavelength of light into corresponding plethysmogram signals, wherein the plethysmogram signals are configured to provide a plethysmogram for each of the first wavelength of light and of the second wavelength of light; determine, based on the plethysmogram signals, a first plethysmogram setpoint; compute a first ratio between the plethysmogram of the first wavelength of light and the plethysmogram of the second wavelength of light; direct, during a second time window, the light emitter to transmit, through the body appendage, the first wavelength of light and the second wavelength of light; direct the light sensor to sense second light signals of the first wavelength of light and of the second wavelength of light; convert the second light signals of the first wavelength of light and of the second wavelength of light into corresponding second plethysmogram signals, wherein the plethysmogram signals are configured to provide a second plethysmogram for each of the first wavelength of light and of the second wavelength of light; determine, based on the second plethysmogram signals, a second plethysmogram setpoint; compute a second ratio between the plethysmogram of the first wavelength of light and the plethysmogram of the second wavelength of light; determine, based on the first and second ratios, a degree of drift between the first and second plethysmogram setpoints; and compute arterial oxygen saturation using the computed ratio.

In a 95th Example, a health monitoring system for arterial oxygen saturation measurement, comprising: a blood pressure cuff in connection with a computational system, wherein the blood pressure cuff is configured to be fitted onto a body appendage, wherein the blood pressure cuff comprises: an inflatable bladder; at least one light emitter, wherein the light emitter is configured to transmit a first wavelength of light and a second wavelength of light through the body appendage; and a light sensor, wherein the light sensor is configured to sense light signals of the first wavelength of light and of the second wavelength of light; a hardware processor; and a non-transitory memory comprising a set of instructions, wherein the set of instructions, when executed by the processor, are configured to direct the processor to: direct the light emitter to transmit the first wavelength of light and the second wavelength of light through the body appendage; direct the light sensor to sense light signals of the first wavelength of light and of the second wavelength of light; convert the light signals of the first wavelength of light and of the second wavelength of light into corresponding plethysmogram signals, wherein the plethysmogram signals are configured to provide a plethysmogram for each of the first wavelength of light and of the second wavelength of light; compute a ratio between the plethysmogram of the first wavelength of light and the plethysmogram of the second wavelength of light; and compute arterial oxygen saturation using the computed ratio; wherein transmitting the first wavelength of light and the second wavelength of light through the body appendage comprises transmitting the first wavelength of light and the second wavelength of light through a portion of the blood pressure cuff or the inflatable bladder.

In a 96th Example, a health monitoring system for arterial oxygen saturation measurement, comprising: a blood pressure cuff in connection with a computational system, wherein the blood pressure cuff is configured to be fitted onto a body appendage, wherein the blood pressure cuff comprises: an inflatable bladder; at least one light emitter configured to transmit a first wavelength of light and a second wavelength of light through the body appendage; and a light sensor configured to sense light signals of the first wavelength of light and of the second wavelength of light; a hardware processor; and a non-transitory memory comprising a set of instructions, wherein the set of instructions, when executed by the processor, are configured to direct the processor to: direct the inflatable bladder to apply a first pressurization to the body appendage, the first pressurization based on a blood pressure of the body appendage; direct the inflatable bladder to apply a second pressurization to the body appendage, the second pressurization lower than the first pressurization; direct, during the second pressurization, the light emitter to transmit the first wavelength of light and the second wavelength of light through the body appendage; direct the light sensor to sense light signals of the first wavelength of light and of the second wavelength of light; convert the light signals of the first wavelength of light and of the second wavelength of light into corresponding plethysmogram signals, wherein the plethysmogram signals are configured to provide a plethysmogram for each of the first wavelength of light and of the second wavelength of light; compute a ratio between the plethysmogram of the first wavelength of light and the plethysmogram of the second wavelength of light; and compute a blood pressure based on the plethysmogram signals.

In a 97th Example, the health monitoring system of any of Examples 89-96, wherein the light signals of the first wavelength are also used to monitor blood pressure via a volume clamp method.

In a 98th Example, the health monitoring system of any of Examples 89-97, wherein the instructions that are configured to direct the processor to transmit a first wavelength of light and a second wavelength of light through a body appendage and that sense light signals of the first wavelength of light and of the second wavelength of light via a light sensor are configured to be performed while an artery within the body appendage is in an unloaded state.

In a 99th Example, the health monitoring system of Example 98, wherein the ratio between the plethysmogram of the first wavelength of light and the plethysmogram of the second wavelength of light is computed using:

$${R}_{Unl} = \frac{( \frac{Δ {d}_{Unl\_ λ 1}}{{DC}_{Unl\_ λ1}} )}{( \frac{Δ {d}_{Unl\_ λ 2}}{{DC}_{Unl\_ λ 2}} )}$$

wherein R_{Unl} is ratio between the plethysmogram of the first wavelength of light and the plethysmogram of the second wavelength computed in an unloaded state; wherein Δd_{Unl_λ1} represents an AC component of the first wavelength when the artery is the unloaded state; Δd_{Unl_λ2} represents an AC component of the second wavelength when the artery is the unloaded state; DC_{Unl_λ1} is a DC component of the first wavelength when the artery is the unloaded state; and DC_{Unl_λ2} is a DC component of the second wavelength when the artery is the unloaded state.

In a 100th Example, the health monitoring system of any of Examples 98-99, wherein the blood pressure cuff comprises an inflatable bladder; wherein the inflatable bladder is configured to apply a pressure onto the body appendage such that the artery therein is in the unloaded state.

In a 101st Example, the health monitoring system of any one of Examples 98-100, wherein the calibration factor is based on a ratio between the plethysmogram of the first wavelength of light and the plethysmogram of the second wavelength of light that is computed while an artery in the body appendage is a loaded state.

In a 102nd Example, the health monitoring system of Example 101, wherein the ratio between the plethysmogram of the first wavelength of light and the plethysmogram of the second wavelength of light is computed using:

$$R = \frac{( \frac{{AC}_{λ1}}{{DC}_{λ1}} )}{( \frac{{AC}_{λ 2}}{{DC}_{λ 2}} )}$$

wherein R is ratio between the plethysmogram of the first wavelength of light and the plethysmogram of the second wavelength; wherein AC_{λ1} is an AC component of the first wavelength; AC_{λ2} is an AC component of the second wavelength; DC_{λ1} is a DC component of the first wavelength; and DC_{λ2} is a DC component of the second wavelength.

In a 103rd Example, the health monitoring system of any of Examples 101-102, wherein the set of instructions is configured to direct the processor to direct the health monitoring system to perform a calibration for determining a plethysmogram setpoint for blood pressure monitoring while the artery in the body appendage is the loaded state.

In a 104th Example, the health monitoring system of any of Examples 101-103, wherein the calibration factor is computed using:

$$Cal\_factor = \frac{{R}_{Loa}}{{R}_{Unl}}$$

where wherein R_{Loa} is ratio between the plethysmogram of the first wavelength of light and the plethysmogram of the second wavelength computed in a loaded state and wherein R_{Unl} is ratio between the plethysmogram of the first wavelength of light and the plethysmogram of the second wavelength computed in an unloaded state.

In a 105th Example, the health monitoring system of Example 104, wherein R_{Loa} is an averaged ratio of two more cardiac cycles.

In a 106th Example, the health monitoring system of Example 104 or 105, wherein R_{Unl} is an averaged ratio of two more cardiac cycles.

In a 107th Example, the health monitoring system of Example 106, wherein the two more cardiac cycles comprise at least one cycle prior to a loaded cardiac cycle and at least one cycle subsequent to the loaded cardiac cycle; wherein R_{Loa} is determined utilizing the loaded cardiac cycle.

In a 108th Example, the health monitoring system of Example 107, wherein the loaded cardiac cycle is utilized to recalibrate a plethysmogram setpoint for blood pressure monitoring.

In a 109th Example, the health monitoring system of any one of Examples 89-108, wherein the set of instructions are further configured to direct the processor to correct the computed ratio for trending plethysmogram signal of the second wavelength of light when the plethysmogram signal of the second wavelength light exhibits trending.

In a 110th Example, the health monitoring system of any one of Examples 89-109, wherein the set of instructions are further configured to direct the processor to correct the computed ratio for variations of pressure provided by the blood pressure cuff.

In a 111th Example, the health monitoring system of any one of Examples 89-110, wherein the first wavelength of light is infrared and the second wavelength of light is red.

In a 112th Example, the health monitoring system of any one of Examples 89-111, wherein the body appendage is: an arm, a finger, a thumb, a wrist, an ankle, a leg, a toe, an ear, or a temple.

In a 113th Example, the health monitoring system of any one of Examples 89-112, further comprising a display, wherein the set of instructions are further configured to direct the processor to display the arterial oxygen saturation.

In a 114th Example, the health monitoring system of any one of Examples 89-113, wherein transmitting the first wavelength of light and the second wavelength of light through the body appendage comprises transmitting the first wavelength of light and the second wavelength of light through a portion of the blood pressure cuff.

In a 115th Example, the health monitoring system of Example 114, wherein the portion of the blood pressure cuff comprises an inflatable bladder.

In a 116th Example, the health monitoring system of any of Examples 89-115, wherein the set of instructions are further configured to determine, based on the plethysmogram signals, a plethysmogram setpoint.

In a 117th Example, a method for measurement of arterial oxygen saturation, comprising: applying a constant pressurization to a body appendage; transmitting, using an emitter of a blood pressure cuff during the constant pressurization, a first wavelength of light and a second wavelength of light through the body appendage; sensing, using a light sensor of the blood pressure cuff, light signals of the first wavelength of light and of the second wavelength of light; converting, using a health monitoring system, the light signals of the first wavelength of light and of the second wavelength of light into corresponding plethysmogram signals, wherein the health monitoring system is in connection with the blood pressure cuff, wherein the plethysmogram signals are configured to provide a plethysmogram for each of the first wavelength of light and of the second wavelength of light; computing, using the health monitoring system, a ratio between the plethysmogram of the first wavelength of light and the plethysmogram of the second wavelength of light; and computing, using the health monitoring system, arterial oxygen saturation using the calibration factor and the computed ratio.

In a 118th Example, a method for measurement of arterial oxygen saturation, comprising: transmitting, using an emitter of a blood pressure cuff through a body appendage, a first wavelength of light at a first time and a second wavelength of light at a second time; sensing, using a light sensor of the blood pressure cuff, light signals of the first wavelength of light and of the second wavelength of light; converting, using a health monitoring system, the light signals of the first wavelength of light and of the second wavelength of light into corresponding plethysmogram signals, wherein the health monitoring system is in connection with the blood pressure cuff, wherein the plethysmogram signals are configured to provide a plethysmogram for each of the first wavelength of light and of the second wavelength of light; computing, using the health monitoring system, a ratio between the plethysmogram of the first wavelength of light and the plethysmogram of the second wavelength of light; and computing, using the health monitoring system, arterial oxygen saturation using the computed ratio.

In a 119th Example, a method for measurement of arterial oxygen saturation, comprising: transmitting, during a first time window through the body appendage and using an emitter of a blood pressure cuff, a first wavelength of light and a second wavelength of light; sensing, using a light sensor of the blood pressure cuff, light signals of the first wavelength of light and of the second wavelength of light; converting, using a health monitoring system, the light signals of the first wavelength of light and of the second wavelength of light into corresponding plethysmogram signals, wherein the health monitoring system is in connection with the blood pressure cuff, wherein the plethysmogram signals are configured to provide a plethysmogram for each of the first wavelength of light and of the second wavelength of light; determining, based on the plethysmogram signals, a first plethysmogram setpoint; computing a first ratio between the plethysmogram of the first wavelength of light and the plethysmogram of the second wavelength of light; transmitting, during a second time window through the body appendage and using the emitter of the blood pressure cuff, the first wavelength of light and the second wavelength of light; sensing, using the light sensor of the blood pressure cuff, second light signals of the first wavelength of light and of the second wavelength of light; converting, using the health monitoring system, the second light signals of the first wavelength of light and of the second wavelength of light into corresponding second plethysmogram signals, wherein the second plethysmogram signals are configured to provide a second plethysmogram for each of the first wavelength of light and of the second wavelength of light; determining, based on the second plethysmogram signals, a second plethysmogram setpoint; computing, using the health monitoring system, a second ratio between the second plethysmogram of the first wavelength of light and the second plethysmogram of the second wavelength of light; determining, based on the first and second ratios, a degree of drift between the first and second plethysmogram setpoints; and computing, using the health monitoring system, arterial oxygen saturation using the computed ratio.

In a 120th Example, a method for measurement of arterial oxygen saturation, comprising: applying, using an inflatable bladder, a first pressurization to a body appendage, the first pressurization based on a blood pressure of the body appendage; applying, using the inflatable bladder, a second pressurization to the body appendage, the second pressurization lower than the first pressurization; transmitting, using an emitter of a blood pressure cuff, a first wavelength of light and a second wavelength of light through a body appendage; sensing, using a light sensor of the blood pressure cuff, light signals of the first wavelength of light and of the second wavelength of light; converting, using a health monitoring system, the light signals of the first wavelength of light and of the second wavelength of light into corresponding plethysmogram signals, wherein the health monitoring system is in connection with the blood pressure cuff, wherein the plethysmogram signals are configured to provide a plethysmogram for each of the first wavelength of light and of the second wavelength of light; computing, using the health monitoring system, a ratio between the plethysmogram of the first wavelength of light and the plethysmogram of the second wavelength of light; calibrating, using the health monitoring system, the computed ratio using a calibration factor; and computing, using the health monitoring system, arterial oxygen saturation using the calibration factor and the computed ratio.

In a 121st Example, the method of any of Examples 117-120, wherein sensing the light signals of the first wavelength of light and of the second wavelength of light comprises applying, using the blood pressure cuff, a pressure to the body appendage such that the artery therein is in the unloaded state.

In a 122nd Example, the method of any of Examples 117-121, further comprising determining, based on the plethysmogram signals of the first wavelength, a blood pressure of the body appendage.

In a 123rd Example, the method of any of Examples 117-122, wherein transmitting the first wavelength of light and the second wavelength of light through the body appendage and sensing light signals of the first wavelength of light and of the second wavelength of light are performed while an artery within the body appendage is in an unloaded state.

In a 124th Example, the method of Example 123, wherein the ratio between the plethysmogram of the first wavelength of light and the plethysmogram of the second wavelength of light is computed using:

$${R}_{Unl} = \frac{( \frac{Δ {d}_{Unl\_ λ 1}}{{DC}_{Unl\_ λ1}} )}{( \frac{Δ {d}_{Unl\_ λ 2}}{{DC}_{Unl\_ λ 2}} )}$$

wherein R_{Unl} is the ratio between the plethysmogram of the first wavelength of light and the plethysmogram of the second wavelength when the artery is in the unloaded state; and wherein: Δd_{Unl_λ1} represents an AC component of the first wavelength when the artery is the unloaded state; Δd_{Unl_λ2} represents an AC component of the second wavelength when the artery is the unloaded state; DC_{Unl_λ1} is a DC component of the first wavelength when the artery is the unloaded state; and DC_{Unl_λ2} is a DC component of the second wavelength when the artery is the unloaded state.

In a 125th Example, the method of any of Examples 123-124, wherein the blood pressure cuff comprises an inflatable bladder and wherein the inflatable bladder applies a pressure onto the body appendage such that the artery therein is in the unloaded state.

In a 126th Example, the method of any one of Examples 117-125, wherein the calibration factor is based on a ratio between the plethysmogram of the first wavelength of light and the plethysmogram of the second wavelength of light for an artery in the body appendage is a loaded state.

In a 127th Example, the method of Example 126, wherein the ratio between the plethysmogram of the first wavelength of light and the plethysmogram of the second wavelength of light is computed using:

$$R = \frac{( \frac{{AC}_{λ1}}{{DC}_{λ1}} )}{( \frac{{AC}_{λ 2}}{{DC}_{λ 2}} )}$$

wherein R is the ratio between the plethysmogram of the first wavelength of light and the plethysmogram of the second wavelength; and wherein: AC_{λ1} is an AC component of the first wavelength in the loaded state; AC_{λ2} is an AC component of the second wavelength in the loaded state; DC_{λ1} is a DC component of the first wavelength in the loaded state; and DC_{λ2} is a DC component of the second wavelength in the loaded state.

In a 128th Example, the method of any of Examples 126-127, further comprising determining, using a second calibration factor, a plethysmogram setpoint for blood pressure monitoring while the artery in the body appendage is the loaded state.

In a 129th Example, the method of any one of Examples 126-128, wherein the calibration factor is computed using:

$$Cal\_factor = \frac{{R}_{Loa}}{{R}_{Unl}}$$

wherein R_{Loa} is a ratio between the plethysmogram of the first wavelength of light and the plethysmogram of the second wavelength in the loaded state, and wherein R_{Unl} is a ratio between the plethysmogram of the first wavelength of light and the plethysmogram of the second wavelength in the unloaded state.

In a 130th Example, the method of Example 129, wherein R_{Loa} is an ratio averaged over two or more cardiac cycles.

In a 131st Example, the method of any of Examples 129-130, wherein Run is a ratio averaged over two or more cardiac cycles.

In a 132nd Example, the method of Example 131, wherein the two or more cardiac cycles comprise at least one cycle prior to a loaded cardiac cycle and at least one cycle subsequent to the loaded cardiac cycle, and wherein R_{Loa} is determined based on the loaded cardiac cycle.

In a 133rd Example, the method of Example 132, further comprising recalibrating, based on the loaded cardiac cycle, the plethysmogram setpoint for blood pressure monitoring.

In a 134th Example, the method of any one of Examples 117-133, further comprising correcting, using the health monitoring system, the computed ratio for a trending plethysmogram signal of the second wavelength of light when the plethysmogram signal of the second wavelength light exhibits trending.

In a 135th Example, the method of any one of Examples 117-134, further comprising correcting, using the health monitoring system, the computed ratio for variations of pressure applied by the blood pressure cuff.

In a 136th Example, the method of any one of Examples 117-135, wherein the first wavelength of light is infrared and the second wavelength of light is red.

In a 137th Example, the method of any one of Examples 117-136, wherein the body appendage comprises at least one of: an arm, a finger, a thumb, a wrist, an ankle, a leg, a toe, an ear, or a temple.

In a 138th Example, the method of any one of Examples 117-137, further comprising displaying the arterial oxygen saturation on a display in connection with the health monitoring system.

In a 139th Example, the method of any one of Examples 117-138, wherein transmitting the first wavelength of light and the second wavelength of light through the body appendage comprises transmitting the first wavelength of light and the second wavelength of light through a portion of the blood pressure cuff.

In a 140th Example, the method of Example 139, wherein the portion of the blood pressure cuff comprises an inflatable bladder.

In a 141st Example, the method of any of Examples 117-140, further comprising determining, based on the plethysmogram signals, a plethysmogram setpoint.

What is claimed is:

1. A health monitoring system for arterial oxygen saturation measurement, comprising:
a blood pressure cuff in connection with a computational system, wherein the blood pressure cuff is configured to be fitted onto a body appendage, wherein the blood pressure cuff comprises:
an inflatable bladder;
a light emitter, wherein the light emitter is configured to transmit a first wavelength of light and a second wavelength of light through the body appendage; and
a light sensor, wherein the light sensor is configured to sense light signals of the first wavelength of light and of the second wavelength of light;
a hardware processor; and
a non-transitory memory comprising a set of instructions,
wherein the set of instructions, when executed by the processor, are configured to direct the processor to:
direct the light emitter to transmit the first wavelength of light and the second wavelength of light through the body appendage;
direct the light sensor to sense light signals of the first wavelength of light and of the second wavelength of light;
convert the light signals of the first wavelength of light and of the second wavelength of light into corresponding plethysmogram signals, wherein the plethysmogram signals are configured to provide a plethysmogram for each of the first wavelength of light and of the second wavelength of light;
compute a ratio between the plethysmogram of the first wavelength of light and the plethysmogram of the second wavelength of light;
calibrate the computed ratio using a calibration factor; and
compute arterial oxygen saturation using the calibration factor and the computed ratio.

2. The health monitoring system of claim 1, wherein the light signals of the first or second wavelength are also used to monitor blood pressure via a volume clamp method.

3. The health monitoring system of claim 1, wherein the instructions that are configured to direct the processor to transmit a first wavelength of light and a second wavelength of light through a body appendage and that sense light signals of the first wavelength of light and of the second wavelength of light via a light sensor are configured to be performed while an artery within the body appendage is in an unloaded state.

4. The health monitoring system of claim 3, wherein the ratio between the plethysmogram of the first wavelength of light and the plethysmogram of the second wavelength of light is computed using:
$${R}_{Unl} = \frac{( \frac{Δ {d}_{Unl\_ λ 1}}{{DC}_{Unl\_ λ1}} )}{( \frac{Δ {d}_{Unl\_ λ 2}}{{DC}_{Unl\_ λ 2}} )}$$
wherein R_{Unl} is ratio between the plethysmogram of the first wavelength of light and the plethysmogram of the second wavelength computed in an unloaded state; wherein Δd_{Unl_λ1} represents an AC component of the first wavelength when the artery is the unloaded state; Δd_{Unl_λ2} represents an AC component of the second wavelength when the artery is the unloaded state; DC_{Unl_λ1} is a DC component of the first wavelength when the artery is the unloaded state; and DC_{Unl_λ2} is a DC component of the second wavelength when the artery is the unloaded state.

5. The health monitoring system of claim 4, wherein the blood pressure cuff comprises an inflatable bladder; wherein the inflatable bladder is configured to apply a pressure onto the body appendage such that the artery therein is in the unloaded state.

6. The health monitoring system of claim 1, wherein the calibration factor is based on a ratio between the plethysmogram of the first wavelength of light and the plethysmogram of the second wavelength of light that is computed while an artery in the body appendage is a loaded state.

7. The health monitoring system of claim 6, wherein the ratio between the plethysmogram of the first wavelength of light and the plethysmogram of the second wavelength of light is computed using:
$$R = \frac{( \frac{{AC}_{λ1}}{{DC}_{λ1}} )}{( \frac{{AC}_{λ 2}}{{DC}_{λ 2}} )}$$
wherein R is ratio between the plethysmogram of the first wavelength of light and the plethysmogram of the second wavelength; wherein AC_{λ1} is an AC component of the first wavelength; AC_{λ2} is an AC component of the second wavelength; DC_{λ1} is a DC component of the first wavelength; and DC_{λ2} is a DC component of the second wavelength.

8. The health monitoring system of claim 7, wherein the set of instructions is configured to direct the processor to direct the health monitoring system to perform a calibration for determining a plethysmogram setpoint for blood pressure monitoring while the artery in the body appendage is the loaded state.

9. The health monitoring system of claim 8, wherein the calibration factor is computed using:
$$Cal\_factor = \frac{{R}_{Loa}}{{R}_{Unl}}$$
where wherein R_{Loa} is ratio between the plethysmogram of the first wavelength of light and the plethysmogram of the second wavelength computed in a loaded state and wherein R_{Unl} is ratio between the plethysmogram of the first wavelength of light and the plethysmogram of the second wavelength computed in an unloaded state.

10. The health monitoring system of claim 9, wherein R_{Loa} is an averaged ratio of two more cardiac cycles.

11. The health monitoring system of claim 9, wherein R_{Unl} is an averaged ratio of two more cardiac cycles.

12. The health monitoring system of claim 11, wherein the two more cardiac cycles comprise at least one cycle prior to a loaded cardiac cycle and at least one cycle subsequent to the loaded cardiac cycle; wherein R_{Loa} is determined utilizing the loaded cardiac cycle.

13. The health monitoring system of claim 12, wherein the loaded cardiac cycle is utilized to recalibrate a plethysmogram setpoint for blood pressure monitoring.

14. The health monitoring system of claim 1, wherein the set of instructions are further configured to direct the processor to correct the computed ratio for trending plethysmogram signal of the second wavelength of light when the plethysmogram signal of the second wavelength light exhibits trending.

15. The health monitoring system of claim 1, wherein the set of instructions are further configured to direct the processor to correct the computed ratio for variations of pressure provided by the blood pressure cuff.

16. The health monitoring system of claim 1, wherein the first wavelength of light is infrared and the second wavelength of light is red.

17. The health monitoring system of claim 1, wherein the body appendage is: an arm, a finger, a thumb, a wrist, an ankle, a leg, a toe, an ear, or a temple.

18. The health monitoring system of claim 1, further comprising a display, wherein the set of instructions are further configured to direct the processor to display the arterial oxygen saturation.

19. The health monitoring system of claim 1, wherein transmitting the first wavelength of light and the second wavelength of light through the body appendage comprises transmitting the first wavelength of light and the second wavelength of light through a portion of the blood pressure cuff.

20. The health monitoring system of claim 19, wherein the portion of the blood pressure cuff comprises an inflatable bladder.

21. A health monitoring system for arterial oxygen saturation measurement, comprising:
a blood pressure cuff in connection with a computational system, wherein the blood pressure cuff is configured to be fitted onto a body appendage, wherein the blood pressure cuff comprises:
an inflatable bladder;
a light emitter comprising an LED, wherein the LED is configured to transmit a first wavelength of light and a second wavelength of light through the body appendage; and
a light sensor, wherein the light sensor is configured to sense light signals of the first wavelength of light and of the second wavelength of light;
a hardware processor; and
a non-transitory memory comprising a set of instructions, wherein the set of instructions, when executed by the processor, are configured to direct the processor to:
direct the LED to transmit the first wavelength of light and the second wavelength of light through the body appendage;
direct the light sensor to sense light signals of the first wavelength of light and of the second wavelength of light;
convert the light signals of the first wavelength of light and of the second wavelength of light into corresponding plethysmogram signals, wherein the plethysmogram signals are configured to provide a plethysmogram for each of the first wavelength of light and of the second wavelength of light;
compute a ratio between the plethysmogram of the first wavelength of light and the plethysmogram of the second wavelength of light; and
compute arterial oxygen saturation using the computed ratio.

22. A health monitoring system for arterial oxygen saturation measurement, comprising:
a blood pressure cuff in connection with a computational system, wherein the blood pressure cuff is configured to be fitted onto a body appendage, wherein the blood pressure cuff comprises:
an inflatable bladder;
at least one light emitter configured to transmit a first wavelength of light and a second wavelength of light through the body appendage; and
a light sensor configured to sense light signals of the first wavelength of light and of the second wavelength of light;
a hardware processor; and
a non-transitory memory comprising a set of instructions, wherein the set of instructions, when executed by the processor, are configured to direct the processor to:
direct the inflatable bladder to apply a constant pressurization to the body appendage;
direct the light emitter to transmit, during the constant pressurization, the first wavelength of light and the second wavelength of light through the body appendage;
direct the light sensor to sense light signals of the first wavelength of light and of the second wavelength of light;
convert the light signals of the first wavelength of light and of the second wavelength of light into corresponding plethysmogram signals, wherein the plethysmogram signals are configured to provide a plethysmogram for each of the first wavelength of light and of the second wavelength of light;
compute a ratio between the plethysmogram of the first wavelength of light and the plethysmogram of the second wavelength of light; and
compute arterial oxygen saturation using the computed ratio.

23. The health monitoring system of claim 22, wherein applying the constant pressurization to the body appendage comprises applying the constant pressurization for at least a full cardiac cycle.

24. The health monitoring system of claim 22, wherein applying the constant pressurization to the body appendage comprises applying a pressure no greater than an ambient pressure.

25. A health monitoring system for arterial oxygen saturation measurement, comprising:
a blood pressure cuff in connection with a computational system, wherein the blood pressure cuff is configured to be fitted onto a body appendage, wherein the blood pressure cuff comprises:
an inflatable bladder;
a light emitter configured to transmit, through the body appendage, a first wavelength of light at a first time and a second wavelength of light at a second time; and
a light sensor configured to sense light signals of the first wavelength of light and of the second wavelength of light;
a hardware processor; and
a non-transitory memory comprising a set of instructions, wherein the set of instructions, when executed by the processor, are configured to direct the processor to:
direct the light emitter to transmit, through the body appendage, the first wavelength of light at the first time and the second wavelength of light at the second time;
direct the light sensor to sense light signals of the first wavelength of light and of the second wavelength of light;
convert the light signals of the first wavelength of light and of the second wavelength of light into corresponding plethysmogram signals, wherein the plethysmogram signals are configured to provide a plethysmogram for each of the first wavelength of light and of the second wavelength of light;
compute a ratio between the plethysmogram of the first wavelength of light and the plethysmogram of the second wavelength of light; and
compute arterial oxygen saturation using the calibration factor and the computed ratio.

26. The health monitoring system of claim 25, wherein a difference between the second time and the first time is less than a full cardiac cycle.

27. A health monitoring system for arterial oxygen saturation measurement, comprising:
a blood pressure cuff in connection with a computational system, wherein the blood pressure cuff is configured to be fitted onto a body appendage, wherein the blood pressure cuff comprises:
an inflatable bladder;
at least one light emitter configured to transmit, through the body appendage, a first wavelength of light and a second wavelength of light; and
a light sensor configured to sense light signals of the first wavelength of light and of the second wavelength of light;
a hardware processor; and
a non-transitory memory comprising a set of instructions, wherein the set of instructions, when executed by the processor, are configured to direct the processor to:
direct, during a first time window, the light emitter to transmit, through the body appendage, the first wavelength of light and the second wavelength of light;
direct the light sensor to sense light signals of the first wavelength of light and of the second wavelength of light;
convert the light signals of the first wavelength of light and of the second wavelength of light into corresponding plethysmogram signals, wherein the plethysmogram signals are configured to provide a plethysmogram for each of the first wavelength of light and of the second wavelength of light;
determine, based on the plethysmogram signals, a first plethysmogram setpoint;
compute a first ratio between the plethysmogram of the first wavelength of light and the plethysmogram of the second wavelength of light;
direct, during a second time window, the light emitter to transmit, through the body appendage, the first wavelength of light and the second wavelength of light;
direct the light sensor to sense second light signals of the first wavelength of light and of the second wavelength of light;
convert the second light signals of the first wavelength of light and of the second wavelength of light into corresponding second plethysmogram signals, wherein the plethysmogram signals are configured to provide a second plethysmogram for each of the first wavelength of light and of the second wavelength of light;
determine, based on the second plethysmogram signals, a second plethysmogram setpoint;
compute a second ratio between the plethysmogram of the first wavelength of light and the plethysmogram of the second wavelength of light;
determine, based on the first and second ratios, a degree of drift between the first and second plethysmogram setpoints; and
compute arterial oxygen saturation using the computed ratio.

28. A health monitoring system for arterial oxygen saturation measurement, comprising:
a blood pressure cuff in connection with a computational system, wherein the blood pressure cuff is configured to be fitted onto a body appendage, wherein the blood pressure cuff comprises:
an inflatable bladder;
at least one light emitter, wherein the light emitter is configured to transmit a first wavelength of light and a second wavelength of light through the body appendage; and
a light sensor, wherein the light sensor is configured to sense light signals of the first wavelength of light and of the second wavelength of light;
a hardware processor; and
a non-transitory memory comprising a set of instructions,
wherein the set of instructions, when executed by the processor, are configured to direct the processor to:
direct the light emitter to transmit the first wavelength of light and the second wavelength of light through the body appendage;
direct the light sensor to sense light signals of the first wavelength of light and of the second wavelength of light;
convert the light signals of the first wavelength of light and of the second wavelength of light into corresponding plethysmogram signals, wherein the plethysmogram signals are configured to provide a plethysmogram for each of the first wavelength of light and of the second wavelength of light;
compute a ratio between the plethysmogram of the first wavelength of light and the plethysmogram of the second wavelength of light; and
compute arterial oxygen saturation using the computed ratio;
wherein transmitting the first wavelength of light and the second wavelength of light through the body appendage comprises transmitting the first wavelength of light and the second wavelength of light through a portion of the blood pressure cuff or the inflatable bladder.

29. A health monitoring system for arterial oxygen saturation measurement, comprising:
a blood pressure cuff in connection with a computational system, wherein the blood pressure cuff is configured to be fitted onto a body appendage, wherein the blood pressure cuff comprises:
an inflatable bladder;
at least one light emitter configured to transmit a first wavelength of light and a second wavelength of light through the body appendage; and
a light sensor configured to sense light signals of the first wavelength of light and of the second wavelength of light;
a hardware processor; and
a non-transitory memory comprising a set of instructions, wherein the set of instructions, when executed by the processor, are configured to direct the processor to:
direct the inflatable bladder to apply a first pressurization to the body appendage, the first pressurization based on a blood pressure of the body appendage;
direct the inflatable bladder to apply a second pressurization to the body appendage, the second pressurization lower than the first pressurization;
direct, during the second pressurization, the light emitter to transmit the first wavelength of light and the second wavelength of light through the body appendage;
direct the light sensor to sense light signals of the first wavelength of light and of the second wavelength of light;
convert the light signals of the first wavelength of light and of the second wavelength of light into corresponding plethysmogram signals, wherein the plethysmogram signals are configured to provide a plethysmogram for each of the first wavelength of light and of the second wavelength of light;
compute a ratio between the plethysmogram of the first wavelength of light and the plethysmogram of the second wavelength of light; and
compute a blood pressure based on the plethysmogram signals.
